(19) United States
(12) Patent Application Publication
(10) Pub. No.: US 2003/0236429 A1
(43) Pub. Date: Dec. 25, 2003

(54) Process for the production of chiral compounds

(75) Inventors: Matthias Gerlach, Brachttal (DE); Claudia Puetz, Dueren (DE); D. Enders, Aachen (DE); Gero Gaube, Aachen (DE)

Correspondence Address: CROWELL & MORING LLP, INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/387,870

(22) Filed: Mar. 14, 2003

Related U.S. Application Data
(63) Continuation of application No. PCT/EP01/10626, filed on Sep. 14, 2001.

(30) Foreign Application Priority Data
Sep. 14, 2000 (DE) 100 45 832.7

(51) Int. Cl. C07C 323/39

(52) U.S. Cl. 560/154

(57) ABSTRACT

A method for producing chiral compounds according to the condition of a 1.4 Michael reaction, and a compound of formula (31).

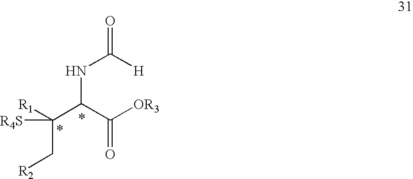

The invention also provides pharmaceutical compositions comprising the compound, and methods for treating pain and other diseases using the pharmaceutical compositions.

### CROSS REFERENCE TO RELATED APPLICATIONS

The present application is a continuation of International Patent Application No. PCT/EP01/10626, filed Sep. 14, 2001, designating the United States of America and published in German as WO 02/22569, the entire disclosure of which is incorporated herein by reference. Priority is claimed based on Federal Republic of Germany Patent Application No. 100 45 832.7, filed Sep. 14, 2000.

### FIELD OF THE INVENTION

The invention relates to a process for the production of chiral compounds under 1,4-Michael addition conditions and to corresponding compounds.

### BACKGROUND AND SUMMARY OF THE INVENTION

Asymmetric Synthesis

Asymmetric synthesis is the central theme of the present application. A carbon atom may form four bonds which are spatially oriented in a tetrahedral shape. If a carbon atom bears four different substituents, there are two possible arrangements which are mirror images of one another. These are known as enantiomers. Chiral molecules (derived from the Greek word cheir meaning hand) have no axis of rotational symmetry They merely differ in one of their physical properties, namely the direction in which they rotate linearly polarized light by an identical amount. In achiral environments, the two enantiomers exhibit the same chemical, biological and physical properties. In contrast, in chiral environments, such as for example the human body, their properties may be very different.

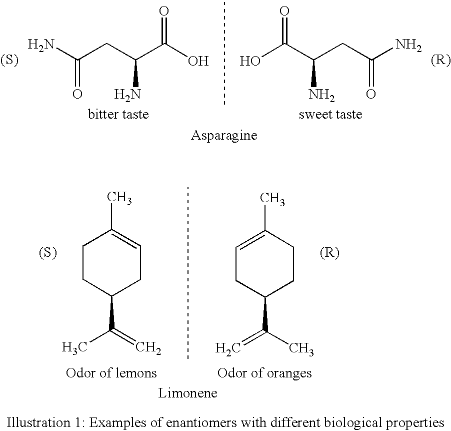

In such environments, the enantiomers each interact differently with receptors and enzymes, such that different physiological effects may occur in nature (see Illustration 1)^{[1]}. For example, the (S) form (S from Latin sinister=left) of asparagine has a bitter flavor, while the (R) form (R from Latin rectus=right) tastes sweet. Limonene, which occurs in citrus fruit, is one everyday example. The (S) form is reminiscent of lemons in odor, while the (R) form smells of oranges. In general, literature references are denoted in the description by Arabic numerals in square brackets which refer to the list of references located between the list of abbreviations and the claims of the instant specification. Where a Roman numeral appears after a literature reference, which is usually cited by the first author's name, the corresponding value (in Arabic numerals) is intended, as it is where the value is not enclosed between square brackets.

Enantiomerically pure substances may be produced by three different methods:

conventional racemate resolution

using natural chiral building blocks (“chiral pool”)

asymmetric synthesis.

Asymmetric synthesis in particular has now come to be of particular significance. It encompasses enzymatic, stoichiometric and also catalytic methods. Asymmetric catalysis is by far the most efficient method as it is possible to produce a maximum quantity of optically active substances using a minimum of chiral catalyst.

The discoveries made by Pasteur^{[2]}, LeBel^{[3]} and van't Hoff^{[4}] aroused interest in optically active substances, because their significance in the complex chemistry of life had been recognized.

D. Enders and W. Hoffmann^{[1]} define asymmetric synthesis as follows:

“An asymmetric synthesis is a reaction in which a chiral grouping is produced from a prochiral grouping in such a manner that the stereoisomericproducts (enantiomers or diastereomers) are obtained in unequal quantities.”

If an asymmetric synthesis is to proceed successfully, diastereomorphic transition states with differing energies must be passed through during the reaction. These determine which enantiomer is formed in excess. Diastereomorphic transition states with different energies may be produced by additional chirality information. This may in turn be provided by chiral solvents, chirally modified reagents or chiral catalysts to form the diastereomorphic transition states.

Sharpless epoxidation is one example of asymmetric catalysis^{[5]}. In this reaction, the chiral catalyst shown in Illustration 2 is formed from the Lewis acid Ti(O-i-Pr)4 and (−)-diethyl tartrate.

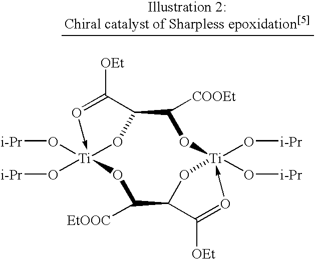

Using this catalyst, allyl alcohols of formula 1 may be epoxidized highly enantioselectively to yield a compound of formula 2 (see Illustration 3), wherein tert.-butyl hydroperoxide is used as the oxidizing agent.

In general, in the description those compounds, in particular those shown in an Illustration or described as a general formula, are usually, but not always, designated and marked with corresponding bold and underlined numerals.

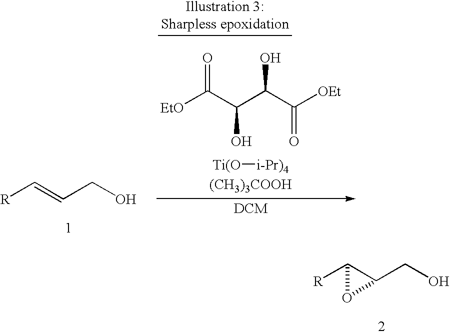

The Sharpless reaction is now a widely used reaction, especially in the chemistry of natural substances. Compounds such as alcohols, ethers or vicinal alcohols may readily be prepared at an optical purity of >90% by nucleophilic ring-opening.

The Michael Reaction

The Michael reaction is of huge significance in organic synthesis and is one of the most important C—C linkage reactions. The reaction has enormous potential for synthesis.

Since there are many different kinds of Michael addition, some examples will be given in the following sections. Particular emphasis is placed here on Michael additions with thiols by asymmetric catalysis.

Conventional Michael addition

The conventional Michael reaction^{[6]}, as shown in Illustration 4, is performed in protic solvents. In this reaction, a carbonyl compound 3 is deprotonated in cc position with a base to form the enolate 4.

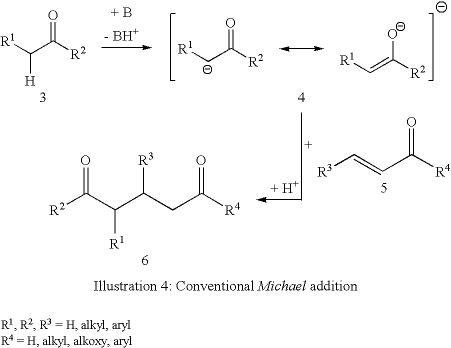

This enolate anion 4 (Michael donor) attacks in the form of a 1,4-addition onto an α,β-unsaturated carbonyl compound 5 (Michael acceptor). After reprotonation, the Michael adduct 6, a 1,5-diketone, is obtained.

The most important secondary reaction which may occur here is the aldol reaction^{[5]}. The enolate anion formed then attacks, not in the β position, but instead directly on the carbonyl oxygen of the Michael acceptor in the form of a 1,2-addition. The aldol reaction is here the kinetically favored process, but this 1,2-addition is reversible. Since the Michael addition is irreversible, the more thermodynamically stable 1,4-adduct is obtained at elevated temperatures.

General Michael Addition

There are now many related 1,4-additions in which the Michael acceptor and/or donor differ(s) from those used in the conventional Michael addition. They are frequently known as “Michael type” reactions or included in the superordinate term “Michael addition.” Today, all 1,4-additions of a nucleophile (Michael donor) onto a C—C multiple bond (Michael acceptor) activated by electron-attracting groups are known as general Michael addition. In this reaction, the nucleophile is 1,4-added onto the activated C—C multiple bond 7 to form the adduct 8 (see Illustration 5)^{[7]}.

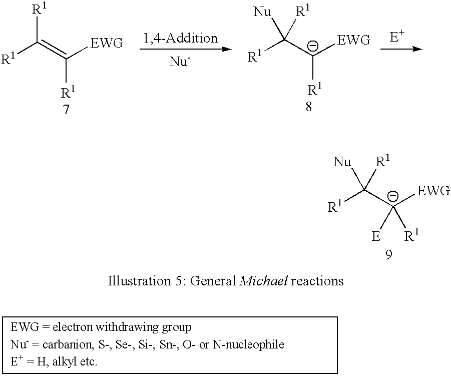

When working in aprotic solvents, the intermediate carbanion 8 may be reacted with electrophiles to form 9 (E=H). If the electrophile is a proton, the reaction is known as a “normal” Michael addition. If, on the other hand, it is a carbon electrophile, it is known as a “Michael tandem reaction” as the 1,4-addition is followed by the second step of the addition of the electrophile^{[8]}.

In addition to the α,β-unsaturated carbonyl compounds, it is also possible to use vinylogous sulfones^{[9]}, sulfoxides^{[10]}, phosphonates^{[11]}and nitroolefins^{[12]}as a Michael acceptor. Nucleophiles which may be used are not only enolates, but also other carbanions together with other heteronucleophiles such as nitrogen^{[13]}, oxygen^{[14]}, silicon^{[15]}, tin^{[16]}, seleniumm^{[17]}and sulfur^{[18]}.

Intramolecular Control of Michael Additions

Intramolecular control is one possible way of introducing asymmetric induction into the Michael addition of thiols on Michael acceptors. In this case, either the Michael acceptor or the thiol already contains a stereogenic center before reaction, the center controlling the stereochemistry of the Michael reaction.

As can be seen in Illustration 6, K. Tomioka et al.^{[19]} have, in a similar manner to Evans with oxazolidinones, used enantiopure N-acrylic acid pyrrolidinones to perform an induced Michael addition with thiols onto 2-alkyl acrylic acids:

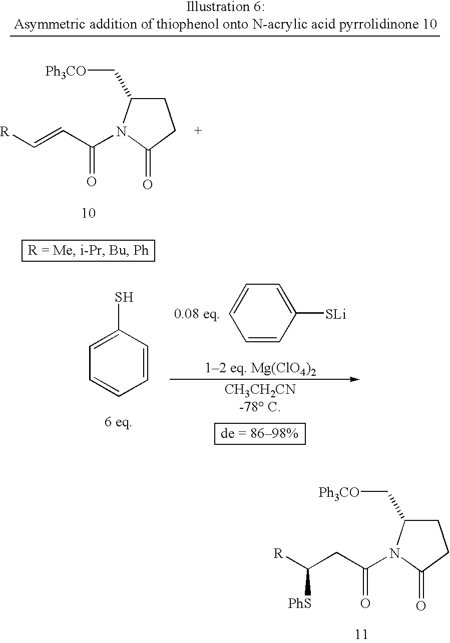

The reaction was predetermined by the (EIZ) geometry of the acrylic pyrrolidinones. Asymmetric induction proceeds by the (R)-triphenylmethoxymethyl group in position 5 of the pyrrolidinone. This bulky “handle” covers the Re side of the double bond during the reaction, so that only the opposite Si side can be attacked. With individual addition of 0.08 equivalents of thiolate or Mg(ClO₄)₂, a de value of up to 70% could be achieved. With combined addition, the de value could even be raised to 98%. The de value is here taken to mean the proportion of pure enantiomer in the product, with the remainder to make up to 100% being the racemic mixture. The ee value has the same definition.

There are many further examples for synthesizing a new stereogenic center, but Michael additions of thiolates with intramolecular control in which two stereogenic centers are formed in a single step are rare.

T. Naito et al.^{[20]} used the oxazolidinones from Evans^{[21]} to introduce the chirality information into the Michael acceptor in a Michael addition in which two new centers were formed (Illustration 7):

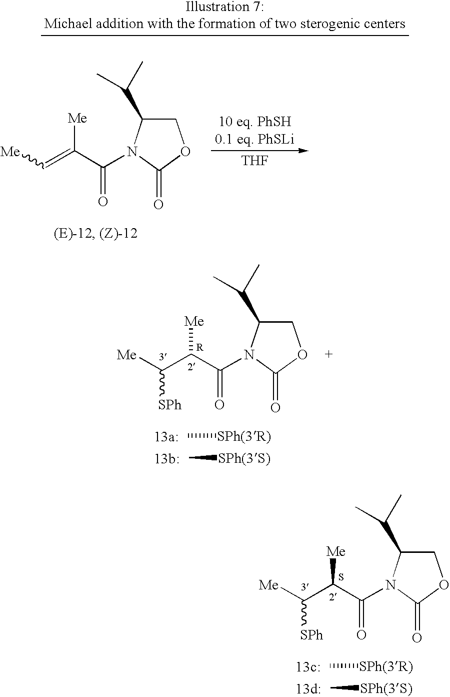

TABLE 1
Test conditions and ratio of the two newly formed centers

| | Yield | Temp. | dr [%] | | | |
|---|---|---|---|---|---|---|
| Educt | [%] | [° C.] | 13a | 13b | 13c | 13d |
| (E)-12 | 84 | RT | >55 | <1 | <1 | >43 |
| (E)-12 | 98 | −50 | >89 | <1 | 4 | 6 |
| (E)-12 | 96 | −50 | >87 | <1 | 4 | 8 |
| (Z)-12 | 95 | −30-−10 | 3 | 4 | <1 | >92 |

In order to achieve elevated diastereomeric (80-86%) and enantiomeric (98%) excesses, a solution of 10 equivalents of thiophenol and 0.1 equivalents of lithium thiophenolate in tetrahydrofuran (THF) was added at low temperatures (−50-−10° C.) to 1 equivalent of the chiral imide 12. Since the methyl group of 12 in 3′ position was exchanged for a phenyl group, diastereomeric excesses of >80% were still obtained in the same reaction. The enantiomeric excesses, however, were still only between 0 and 50%. The stereocenter in 2′ position could be selectively controlled in this case too, but only low levels of selectivity could be achieved on the center in 3′ position.

Michael Addition Catalyzed by Chiral Bases

Michael addition of thiols onto α,β-unsaturated carbonyl compounds catalyzed by bases such as triethylamine or piperidine has long been known^{[22]}. When achiral educts are used, however, enantiopure bases are required in order to obtain optically active substances.

T. Mukaiyama et al.^{[23]} investigated the use of hydroxyproline derivatives 14 as a chiral catalyst:

TABLE 2
Chiral hydroxyproline bases

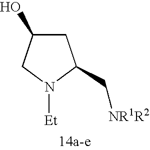

| No. | R1 | R2 |
|---|---|---|
| 14a | H | Phenyl |
| 14b | H | Cyclohexyl |
| 14c | H | 1,5-Dimethylphenyl |
| 14d | H | 1-Naphthyl |
| 14e | Me | Phenyl |

The addition of thiophenol (0.8 equivalents) and cyclohexanone (1 equivalent) was investigated with the hydroxyproline derivatives 14a-e (0.008 equivalents) in toluene. It was found that, when using 14d, an ee value of 72% could be achieved.

Many alkaloids were likewise tested for chiral base catalysis. Particularly frequent and extensive use was made of cinchona alkaloids^{[24],[25]} and ephedrine alkaloids.

H. Wynberg^{[26]} accordingly carried out very exhaustive testing of the Michael addition of thiophenol onto α,β-unsaturated cyclohexanones with cinchona and ephedrine alkaloids (see Illustration 8) for catalysis and control:

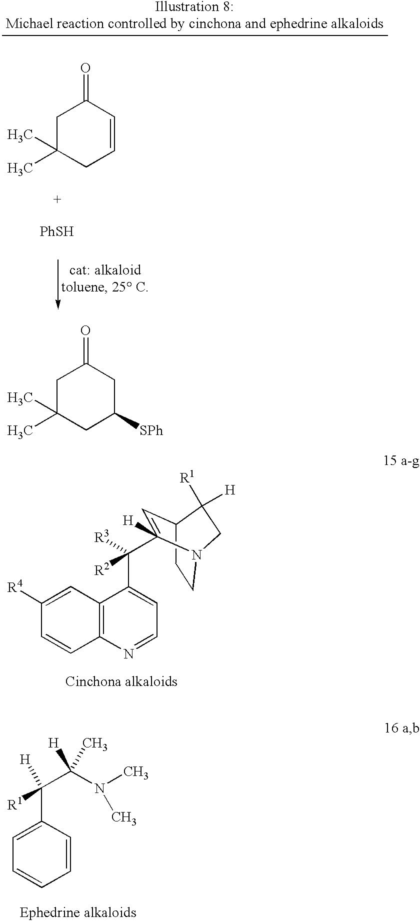

TABLE 3
Enantiomeric excess when using various alkaloids in Michael addition

| No. | Name | R1 | R2 | R3 | R4 | ee[%] |
|---|---|---|---|---|---|---|
| 15a | Quinine | C2H3 | OH | H | OCH3 | 44 |
| 15b | Cinchonidine | C2H3 | OH | H | H | 62 |
| 15c | Dihydroquinine | C2H5 | OH | H | OCH3 | 35 |
| 15d | Epiquinine | C2H3 | H | OH | OCH3 | 18 |
| 15e | Acetylquinine | C2H3 | OAc | H | OCH3 | 7 |
| 15f | Deoxycinchonidine | C2H3 | H | H | H | 4 |
| 15g | Epichlorocinchonidine | C2H3 | H | Cl | H | 3 |
| 16a | (-)-N-Methylephedrine | OH | — | — | — | 29 |
| 16b | N,N-Dimethylamphetamine | H | — | — | — | 0 |

As is clear from Table 3 even a slight change in the residues R1-R4 in the alkaloid 15, 16 brought about a distinct change in the enantiomeric excess. This means that the catalyst must be tailored to the educts. If, for example, p-methylthiophenol was used instead of thiophenol, a distinct worsening of the enantiomeric excess could be observed with the same catalyst.

Michael Addition with Chiral Lewis Acid Catalysis

Simple catalysis of the Michael addition of thiols onto Michael acceptors by simple Lewis acids, such as TiCl4, sometimes with good yield, has long been known^{[27]}.

There are several examples of catalysis by chiral Lewis acids, in which, as also in the case of intramolecular control (section 1.2.3), N-acrylic acid oxazolidinones were used. However, this time, these do not contain a chiral center. The further carbonyl group of the introduced oxazolidinone ring is required to chelate the metal atom of the chiral Lewis acid→17. The Lewis acid 18 was used by D. A. Evans for the addition of silyl enol ethers onto the N-acrylic acid oxazolidinone 17+ Lewis acid complex 18 with diastereomeric excesses of 80-98% and enantiomeric excesses of 75-99% (see Illustration 9)^{[28]}.

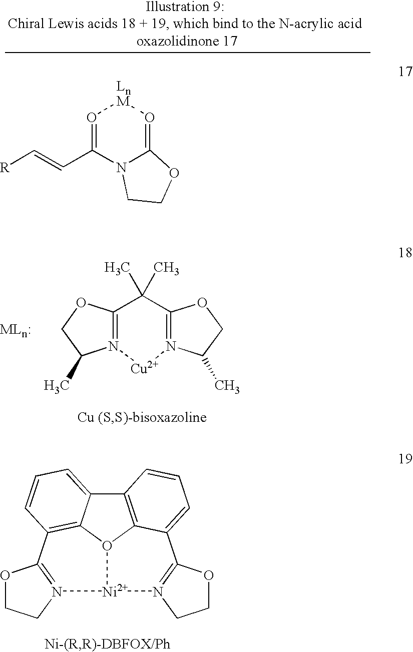

The Lewis acid Ni-(R,R)-DBFOX/Ph (DBFOX/Ph=4,6-dibenzofurandiyl-2,2′-bis-(4-phenyloxazoline)) 19 was used by S. Kanemasa for the addition of thiols onto 17^{[29]}. He achieved enantiomeric excesses of up to 97% with good yields.

In many instances, 1,1-binaphthols (binol) were also bound to metal ions in order to form chiral Lewis acids (see Illustration 10). B. L. Fernnga^{[30]} accordingly synthesized an LiAl binol complex 20, which he used in a Michael addition of X-nitro esters onto α,β-unsaturated ketones. At −20° C. in THF, when using 10 mol % of LiAl binol 20, he obtained Michael adducts with an ee of up to 71%.

Shibasaki^{[31]}uses the NaSm binol complex 21 in the Michael addition of thiols onto α,β-unsaturated acyclic ketones. At −40° C., he obtained Michael adducts with enantiomeric excesses of 75-93%.

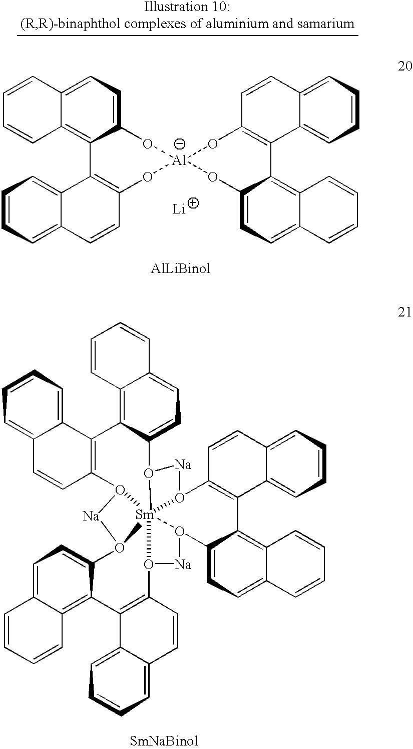

On addition of the Michael donor and acceptor, these chiral Lewis acids form a diastereomorphic transition state, by means of which the reaction is then controlled.

Control of Michael Addition by Complexation of the Lithiated Nucleophile

Another way of controlling the attack of a nucleophile (Michael donor) in a reaction is to complex the lithiated nucleophile by an external chiral ligand.

Tomioka et al.^{[32]} have tested many external chiral ligands for controlled attack of organometallic compounds in various reactions, such as aldol additions, alkylations of enolates, Michael additions, etc. Illustration 11 shows several examples of enantiomerically pure compounds with which Tomioka complexed organometallic compounds.

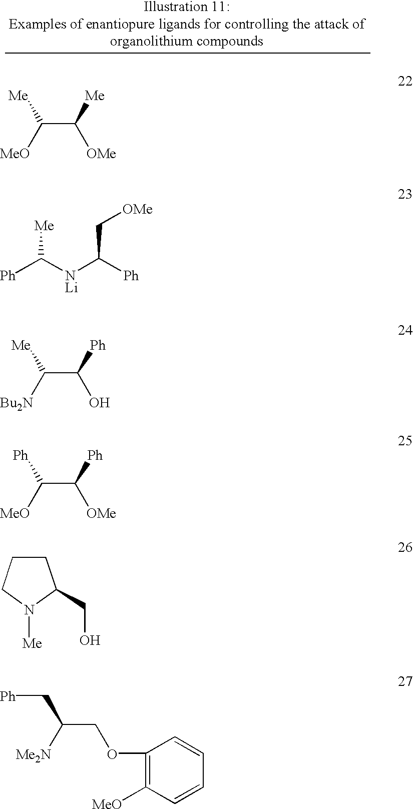

For example, using dimethyl ether 22, he controlled the aldol addition of dimethylmagnesium onto benzaldehyde and obtained an enantiomeric excess of 22%. In contrast, with lithium amide 23, he achieved an enantiomeric excess of 90% in the addition of BuLi onto benzaldehyde. With 24, he achieved enantiomeric excesses of 90% in the addition of diethylzinc onto benzaldehyde. Using the proline derivative 26, he controlled the addition of organometallic compounds onto Michael systems with enantiomeric excesses of up to 90%. Using 27, he was only able to achieve an ee of 50% in the alkylation of cyclic enamines.

Tomioka subsequently extended his synthesis, by using not only organolithium compounds, but also lithium thiolates^{[33]}. He used chiral dimethyl ethers such as for example 25, sparteine or chiral diethers for this purpose. This latter is related to 27 and, thanks to a phenyl substituent in 2 position, has a further chiral center. In a Michael addition of lithium thiolates onto methyl acrylates enantiomeric excesses of 90% could be achieved for these chiral diethers, but only of 6% for 25.

If it is considered that in every case the chiral compounds are used in only catalytic quantities of 5-10 mol %, some of these enantiomeric excesses should be deemed very good.

Tomioka proposed the concept of the asymmetric oxygen atom for the dimethyl ethers 28 in nonpolar solvents^{[34]}.

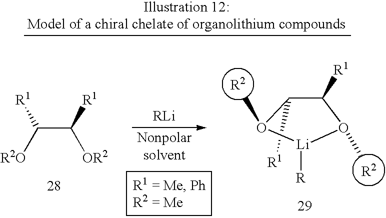

As shown in Illustration 12, due to steric effects, the residues of 28 in the complex 29 are in all-trans position. Thanks to the asymmetric carbon atoms in the ethylene bridge, the adjacent oxygen atoms become asymmetric centers. According to X-ray structural analysis, these oxygen atoms, which chelate the lithium, in 29 are tetrahedrally coordinated. The chirality information is thus provided directly adjacent to the chelating lithium atom by the bulky residue R².

### DESCRIPTION OF THE INVENTION

The object of the invention was in general to develop an asymmetric synthesis under Michael addition conditions, which synthesis avoids certain disadvantages of the prior art and provides good yields.

Specifically, the object was to provide a simple synthetic pathway for producing 2-formylamino-3-dialkyl acrylic acid esters 30 and for separating from one another the (E,Z) mixtures of the synthesized acrylic acid esters 30. A further object was, on the basis of the synthesized Michael acceptor 30, to find a pathway for Michael addition with thiols. It would first be necessary to find a Lewis acid catalyst for this addition, which catalyst can subsequently be provided with chiral ligands for control (see Illustration 13), so directly determining the diastereomeric and enantiomeric excesses of the Michael adducts 31.

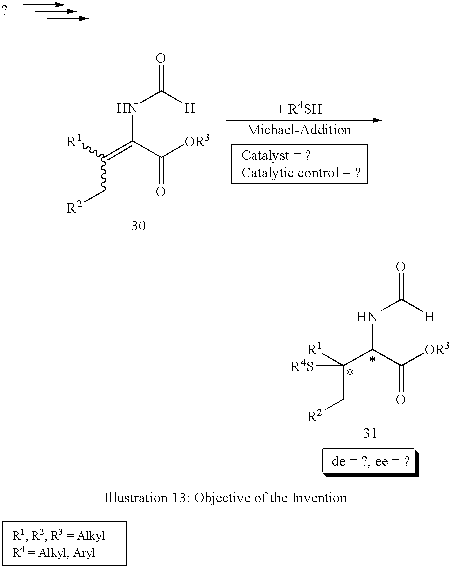

The invention accordingly generally provides a process for the production of a compound of formula 9

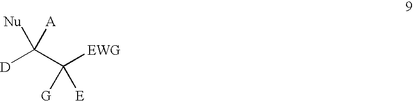

wherein a compound of formula 7 is reacted under suitable 1,4-Michael addition conditions with a nucleophile Nu⁻ according to the following reaction scheme

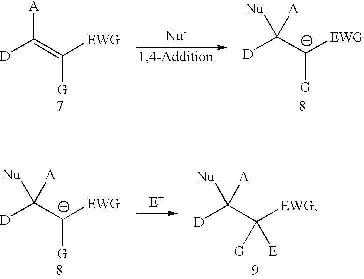

in which the residues

A, D and G are mutually independently identical or different and represent any desired substituents,

E is H or alkyl,

Nu is a C-, S-, Se-, Si-, Si-, O- or N-nucleophile,

and EWG denotes an electron-attracting group,

wherein the reaction conditions are selected such that the stereoisomeric, in particular enantiomeric and/or diastereomeric, products are obtained in unequal quantities. It is particularly preferred if the nucleophile Nu⁻ is an S-nucleophile.

The invention specifically provides a process for the production of a compound of formula 31

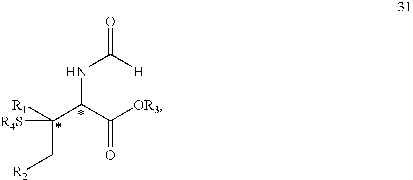

in which

R1, R2 and R3 are, independent of each other, C₁₋₁₀ alkyl, saturated or unsaturated, branched or unbranched, mono- or polysubstituted or unsubstituted;

* indicates a stereoselective center; and

R4 is:

C1-10 alkyl, saturated or unsaturated, branched or unbranched, mono- or polysubstituted or unsubstituted; C3-8 cycloalkyl, saturated or unsaturated, unsubstituted or mono- or polysubstituted; aryl or heteroaryl, in each case unsubstituted or mono- or polysubstituted; or aryl, C3-8 cycloalkyl or heteroaryl, in each case unsubstituted or mono- or polysubstituted, attached via saturated or unsaturated C1-3 alkyl.

According to the process of the invention, a compound of formula 30, is reacted under Michael addition conditions with a compound of the formula R4SH, in accordance with reaction I below:

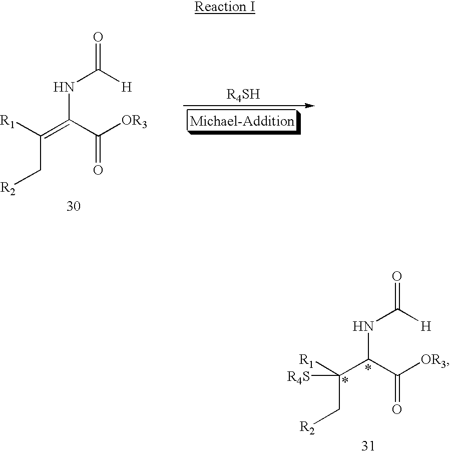

wherein the compounds of the formula R4SH are used as lithium thiolates or are converted into lithium thiolates during or before reaction I, Chiral catalysts, chosen from: chiral auxiliary reagents, in particular the diether (S, S)-1,2-dimethoxy-1,2-diphenylethane; Lewis acids; and/or Bronsted bases or combinations thereof, are optionally used, the products are optionally then hydrolyzed with bases, in particular NaOH, and optionally purified, preferably by column chromatography.

For the purposes of the present invention alkyl or cycloalkyl residues are taken to mean saturated and unsaturated (but not aromatic), branched, unbranched and cyclic hydrocarbons, which may be unsubstituted or mono- or polysubstituted. C₁₋₂ alkyl here denotes C1 or C2 alkyl, C₁₋₃ alkyl denotes C₁, C₂ or C₃ alkyl, C₁₋₄ alkyl denotes C₁, C₂, C₃ or C₄ alkyl, C₁₋₅ alkyl denotes C₁, C₂, C₃, C₄ or C₅ alkyl, C₁₋₆ alkyl denotes C₁, C₂, C₃, C₄, C₅ or C₆ alkyl, C₁₋₇ alkyl denotes C₁, C₂, C₃, C₄, C₅, C₆ or C₇ alkyl, C₁₋₈ alkyl denotes C₁, C₂, C₃, C₄, C₅, C₆, C₇ or C₈ alkyl, C₁₋₄₀ alkyl denotes C₁, C₂, C₃, C₄, C₅, C₆, C₇, C₈, Cg or C₁₋₀ alkyl and C₁₋₁₈ alkyl denotes C₁, C₂, C₃, C₄, C₅, C₆, C₇, C₈, C₉, C₁₀, C₁₁, C₁₂, C₁₃, C₁₄, C₁₅, C₁₆, C₁₇ or C₁₈ alkyl. C₃₋₄ cycloalkyl furthermore denotes C₃ or C₄ cycloalkyl, C₃₋₅ cycloalkyl denotes C₃, C₄ or C₅ cycloalkyl, C₃₋₆ cycloalkyl denotes C₃, C₄, C₅ or C₆ cycloalkyl, C₃₋₇ cycloalkyl denotes C₃, C₄, C₅, C₆ or C₇ cycloalkyl, C₃₋₈ cycloalkyl denotes C₃, C₄, C₅, C₆, C₇ or C₈ cycloalkyl, C₄₋₅ cycloalkyl denotes C₄ or C₅ cycloalkyl, C₄₋₆ cycloalkyl denotes C₄, C₅ or C₆ cycloalkyl, C₄₋₇ cycloalkyl denotes C₄, C₅, C₆ or C₇ cycloalkyl, C₅₋₆ cycloalkyl denotes C₅ or C₆ cycloalkyl and C₅₋₇ cycloalkyl denotes C₅, C₆ or C₇ cycloalkyl. With regard to cycloalkyl, the term also includes saturated cycloalkyls in which one or 2 carbon atoms are replaced by a heteroatom S, N or O. The term cycloalkyl in particular, however, also includes mono- or polyunsaturated, preferably monounsaturated, cycloalkyls without a heteroatom in the ring, provided that the cycloalkyl does not constitute an aromatic system. The alkyl or cycloalkyl residues are preferably methyl, ethyl, vinyl (ethenyl), propyl, allyl (2-propenyl), 1-propynyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, cyclopropyl, 2-methylcyclopropyl, cyclopropylmethyl, cyclobutyl, cyclopentyl, cyclopentylmethyl, cyclohexyl, cycloheptyl, cyclooctyl, as well as adamantyl, CHF₂, CF₃ or CH₂OH and pyrazolinone, oxopyrazolinone, [1,4]-dioxane or dioxolane.

In relation to alkyl and cycloalkyl, it is here understood that, unless explicitly stated otherwise, for the purposes of the present invention, substituted means the substitution at least one hydrogen residue by F, Cl, Br, I, NH₂, SH or OH, wherein “polysubstituted” residues should be taken to mean that substitution is performed repeatedly both on different and the same C atoms with identical or different substituents, for example three times on the same C atom as in case of CF₃ or on different sites as in the case of —CH(OH)—CH═CH—CHCl₂. Particularly preferred substituents are here F, Cl and OH. With regard to cycloalkyl, the hydrogen residue may also be replaced by OC₁₋₃ alkyl or C₁₋₃ alkyl (in each case mono- or polysubstituted or unsubstituted), in particular methyl, ethyl, n-propyl, i-propyl, CF₃, methoxy or ethoxy.

The term (CH₂)₃₋₆ should be taken to mean —CH₂—CH₂—CH₂—, —CH₂—CH₂—CH₂—CH₂—, —CH₂—CH₂—CH₂—CH₂—CH₂— and CH₂—CH₂—CH₂—CH₂—CH₂—CH₂—, while (CH₂)₁₄ should be taken to mean —CH₂—, —CH₂—CH₂—, —CH₂—CH₂—CH₂— and —CH₂—CH₂—CH₂—CH₂— and (CH₂)₄₋₅ should be taken to mean CH₂—CH₂—CH₂—CH₂— and —CH₂—CH₂—CH₂—CH₂—CH₂—, etc.

An aryl residue is taken to mean ring systems comprising at least one aromatic ring, but without a heteroatom in any of the rings. Examples are phenyl, naphthyl, fluoranthenyl, fluorenyl, tetralinyl or indanyl, in particular 9H fluorenyl or anthacenyl residues, which may be unsubstituted or mono- or polysubstituted.

A heteroaryl residue is taken to mean heterocyclic ring systems comprising at least one unsaturated ring, which contain one or more heteroatoms from the group of nitrogen, oxygen and/or sulfur and may also be mono- or polysubstituted. Examples from the group of heteroaryls which may be mentioned are furan, benzofuran, thiophene, benzothiophene, pyrrole, pyridine, pyrimidine, pyrazine, quinoline, isoquinoline, phthalazine, benzo-1,2,5-thiadiazole, benzothiazole, indole, benzotriazole, benzodioxolane, benzodioxane, carbazole, indole and quinazoline.

In relation to aryl and heteroaryl, substituted is taken to mean the substitution of the aryl or heteroaryl with R²³, OR²³, a halogen, preferably F and/or Cl, a CF₃, a CN, an NO₂, an NR²⁴R²⁵, a C₁₋₆ alkyl (saturated), a C₁₋₆ alkoxy, a C₃₋₈ cycloalkoxy, a C₃₋₈ cycloalkyl or a C₂₋₆ alkylene.

The residue R²³ here denotes H, a C₁₋₁₀ alkyl, preferably a C₁₋₆ alkyl, an aryl or heteroaryl or an aryl or heteroaryl residue attached via a C₁₋₃ alkylene group, wherein these aryl or heteroaryl residues may not themselves be substituted with aryl or heteroaryl residues, the residues R²⁴ and R²⁵, identical or different, denote H, a C₁₋₁₀ alkyl, preferably a C₁₋₆ alkyl, an aryl, a heteroaryl or an aryl or heteroaryl attached via a C₁₋₃ alkylene group, wherein these aryl and heteroaryl residues may not themselves be substituted with aryl or heteroaryl residues, or the residues R24 and R25 together mean CH₂CH₂OCH₂CH₂, CH₂CH₂NR²⁶CH₂CH₂ or (CH₂)₃₋₆, and

the residue R²⁶ denotes H, a C₁₋₁₀ alkyl, preferably a C₁₋₆ alkyl, an aryl or heteroaryl residue or denotes an aryl or heteroaryl residue attached via a C₁₋₃ alkylene group, wherein these aryl or heteroaryl residues may not themselves be substituted with aryl or heteroaryl residues.

In a preferred embodiment of the process according to the invention, the compounds of the formula R₄SH are used as lithium thiolates or are converted into lithium thiolates during or before reaction I.

In a preferred embodiment of the process according to the invention, butyllithium (BuLi) is used before reaction I to convert the compounds of the formula R4SH into lithium thiolates, preferably in an equivalent ratio of BuLi:R4SH of between 1:5 and 1:20, in particular 1:10, and is reacted with R4SH and/or the reaction proceeds at temperatures of <0° C. and/or in an organic solvent, in particular toluene, ether, THF or dichloromethane (DCM), especially THE

In a preferred embodiment of the process according to the invention, at the beginning of reaction I, the reaction temperature is at temperatures of <0° C., preferably at between −70 and −80° C., in particular −78° C., and, over the course of reaction I, the temperature is adjusted to room temperature, or the reaction temperature at the beginning of reaction I is at temperatures of ≦0° C., preferably at between −30 and −20° C., in particular −25° C., and, over the course of reaction I, the temperature is adjusted to between −20° C. and −10° C., in particular −15° C.

In a preferred embodiment of the process according to the invention, reaction I proceeds in an organic solvent, preferably toluene, ether, THF or DCM, in particular in THF, or a nonpolar solvent, in particular in DCM or toluene.

In a preferred embodiment of the process according to the invention, the diastereomers are separated after reaction I, preferably by preparative HPLC or crystallization, in particular using the solvent pentane/ethanol (10:1) and cooling.

In a preferred embodiment of the process according to the invention, separation of the enantiomers proceeds before separation of the diastereomers.

In a preferred embodiment of the process according to the invention, R¹ means C₁₋₆ alkyl, saturated or unsaturated, branched or unbranched, mono- or polysubstituted or unsubstituted, and R² means C₂₋₉ alkyl, saturated or unsaturated, branched or unbranched, mono- or polysubstituted or unsubstituted;

preferably

R¹ means C₁₋₂ alkyl, mono- or polysubstituted or unsubstituted, in particular methyl or ethyl, and

R² means C₂₋₉ alkyl, preferably C₂₋₇ alkyl, saturated or unsaturated, branched or unbranched, mono- or polysubstituted or unsubstituted, in particular ethyl, propyl, n-propyl, i-propyl, butyl, n-butyl, i-butyl, tert.-butyl, pentyl, hexyl or heptyl;

in particular

R¹ means methyl and R² means n-butyl.

In a preferred embodiment of the process according to the invention, R³ is C₁₋₃ alkyl, saturated or unsaturated, branched or unbranched, mono- or polysubstituted or unsubstituted, preferably methyl or ethyl.

In a preferred embodiment of the process according to the invention, R⁴ is C₁₋₆ alkyl, saturated or unsaturated, branched or unbranched, mono- or polysubstituted or unsubstituted; phenyl or thiophenyl, unsubstituted or monosubstituted (preferably with OCH₃, CH₃, OH, SH, CF₃, F, Cl, Br or I); or phenyl attached via saturated CH₃, unsubstituted or monosubstituted (preferably with OCH₃, CH₃, OH, SH, CF₃, F, Cl, Br or I);

R⁴ is preferably C₁₋₆ alkyl, saturated, unbranched and unsubstituted, in particular methyl, ethyl, propyl, n-propyl, i-propyl, butyl, n-butyl, i-butyl, tert.-butyl, pentyl or hexyl; phenyl or thiophenyl, unsubstituted or monosubstituted (preferably with OCH₃, CH₃, OH, SH, CF₃, F, Cl, Br or I); or phenyl attached via saturated CH₃, unsubstituted or monosubstituted (preferably with OCH₃, CH₃, OH, SH, CF₃, F, Cl, Br or I),

in particular R⁴ is selected from among methyl, ethyl or benzyl, unsubstituted or monosubstituted (preferably with OCH₃, CH₃, OH, SH, CF₃, F, Cl, Br or I).

In a preferred embodiment of the process according to the invention, the thiolate is used stoichiometrically, chlorotrimethylsilane (TMSCl) is used and/or a chiral proton donor R*-H is then used,

or

compound 30 is modified before reaction I with a sterically demanding (large) group, preferably a t-Butyldimethylsiloxy (TBDMS) group.

In a preferred embodiment of the process according to the invention, the compound of formula 31 is 3-ethylsulfanyl-2-formylamino-3-methyloctanoic acid ethyl ester or 3-benzylsulfanyl-2-formylamino-3-methyloctanoic acid ethyl ester, the compound of formula 30 is 2-formylamino-3-methyl-2-octenoic acid ethyl ester and R₄SH is ethyl mercaptan or benzyl mercaptan.

The other conditions and embodiments of Michael addition, are explained below.

The invention also provides a compound of formula 31

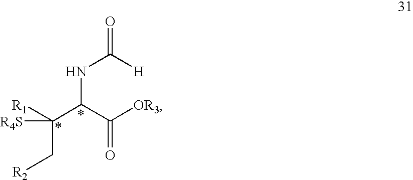

in which

R1, R2 and R3 are independently C₁₋₁₀ alkyl, saturated or unsaturated, branched or unbranched, mono- or polysubstituted or unsubstituted;

* indicates a stereoselective center, and

R⁴ is:

C₁₋₁₀ alkyl, saturated or unsaturated, branched or unbranched, mono- or polysubstituted or unsubstituted; C₃₋₈ cycloalkyl, saturated or unsaturated, unsubstituted or mono- or polysubstituted; aryl or heteroaryl, in each case unsubstituted or mono- or polysubstituted; or aryl, C₃₋₈ cycloalkyl or heteroaryl, in each case unsubstituted or mono- or polysubstituted, attached via saturated or unsaturated C₁₋₃ alkyl;

in the form of the racemates, enantiomers, diastereomers thereof, in particular mixtures of the enantiomers or diastereomers thereof or of a single enantiomer or diastereomer; in the form of their physiologically acceptable acidic or basic salts or salts with cations or bases or with anions or acids; or in the form of the free acids or bases.

The term salt should be taken to mean any form of the active substance according to the invention, in which the latter assumes ionic form or bears a charge and is coupled with a counterion (a cation or anion) or is in solution. These should also be taken to mean complexes of the active substance with other molecules and ions, in particular complexes which are complexed by means of ionic interactions.

For the purposes of the present invention, a physiologically acceptable salt with cations or bases is taken to mean salts of at least one of the compounds according to the invention, usually a (deprotonated) acid, as the anion with at least one, preferably inorganic, cation, which is physiologically acceptable, in particular for use in humans and/or mammals. Particularly preferred salts are those of the alkali and alkaline earth metals, as are those with NH₄⁺, most particularly (mono-) or (di-) sodium, (mono-) or (di-)potassium, magnesium or calcium salts.

For the purposes of the present invention, a physiologically acceptable salt with anions or acids is taken to mean salts of at least one of the compounds according to the invention, usually protonated, for example on the nitrogen, as the cation with at least one anion, which is physiologically acceptable, in particular for use in humans and/or other mammals. In particular, for the purposes of the present invention, the physiologically acceptable salt is taken to be the salt formed with a physiologically acceptable acid, namely salts of the particular active substance with inorganic or organic acids which are physiologically acceptable, in particular for use in humans and/or other mammals. Examples of physiologically acceptable salts of certain acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1-dioxo-1,2-dihydro-1,6-benzo[d]isothiazol-3-one (saccharinic acid), monomethylsebacic acid, 5-oxo-proline, hexane-1-sulfonic acid, nicotinic acid, 2-, 3- or 4-aminobenzoic acid, 2,4,6-trimethylbenzoic acid, α-lipoic acid, acetylglycine, acetylsalicylic acid, hippuric acid and/or aspartic acid. The hydrochloride salt is particularly preferred.

In a preferred form of the compounds according to the invention, R¹ means C₁₋₆ alkyl, saturated or unsaturated, branched or unbranched, mono- or polysubstituted or unsubstituted, and R² means C₂₋₉ alkyl, saturated or unsaturated, branched or unbranched, mono- or polysubstituted or unsubstituted,

preferably

R¹ means C₁₋₂ alkyl, mono- or polysubstituted or unsubstituted, in particular methyl or ethyl and R² means C₂₋₉ alkyl, preferably C₂₋₇ alkyl, saturated or unsaturated, branched or unbranched, mono- or polysubstituted or unsubstituted, in particular ethyl, propyl, n-propyl, i-propyl, butyl, n-butyl, i-butyl, tert.-butyl, pentyl, hexyl or heptyl;

in particular

R¹ means methyl and R² means n-butyl.

In a preferred form of the compounds according to the invention, R³ is C₁₋₃ alkyl, saturated or unsaturated, branched or unbranched, mono- or polysubstituted or unsubstituted, preferably methyl or ethyl.

In a preferred form of the compounds according to the invention, R⁴ is C₁₋₆ alkyl, saturated or unsaturated, branched or unbranched, mono- or polysubstituted or unsubstituted; phenyl or thiophenyl, unsubstituted or monosubstituted (preferably with OCH₃, CH₃, OH, SH, CF₃, F, Cl, Br or I); or phenyl attached via saturated CH₃, unsubstituted or monosubstituted (preferably with OCH₃, CH₃, OH, SH, CF₃, F, Cl, Br or I);

R⁴ is preferably C₁₋₆ alkyl, saturated, unbranched and unsubstituted, in particular methyl, ethyl, propyl, n-propyl, i-propyl, butyl, n-butyl, i-butyl, tert.-butyl, pentyl or hexyl; phenyl or thiophenyl, unsubstituted or monosubstituted (preferably with OCH₃, CH₃, OH, SH, CF₃, F, Cl, Br or I); or phenyl attached via saturated CH₃, unsubstituted or monosubstituted (preferably with OCH₃, CH₃, OH, SH, CF₃, F, Cl, Br or I),

in particular R⁴ is methyl, ethyl or benzyl, unsubstituted or monosubstituted (preferably with OCH₃, CH₃, OH, SH, CF₃, F, Cl, Br or I).

In a preferred form, the compound is selected from among

3-ethylsulfanyl-2-formylamino-3-methyloctanoic acid ethyl ester, or

3-benzylsulfanyl-2-formylamino-3-methyloctanoic acid ethyl ester.

The compounds according to the invention are pharmacologically active, in particular as analgesics, and toxicologically safe. Accordingly, the invention also provides pharmaceutical preparations containing the compounds according to the invention optionally together with suitable additives and/or auxiliary substances and/or optionally further active substances. The invention furthermore provides a process or the use of the compounds according to the invention for the production of a pharmaceutical preparation for the treatment of pain, in particular of neuropathic, chronic or acute pain, of epilepsy and/or migraine, together with corresponding treatment methods.

The following Examples are, intended to illustrate the invention, but without restricting its scope.

### EXAMPLES

### Example 1

Synthetic Pathway

The target molecule 32/33 is to be prepared by a Michael addition. Illustration 14 shows the retrosynthetic analysis of the educt 34 required for this approach:

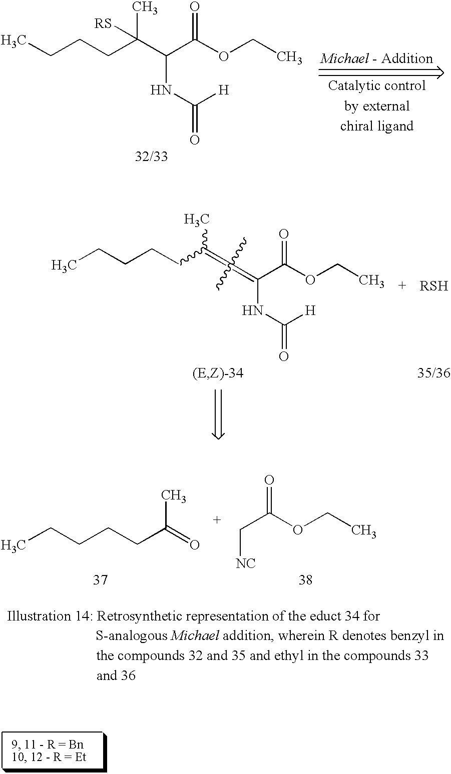

The 2-formylaminoacrylic acid ester 34 is to be produced in an olefination reaction from the ketone 37 and from isocyanoacetic acid ethyl ester (33).

Illustration 15 shows the synthetic pathway for the preparation of 38:

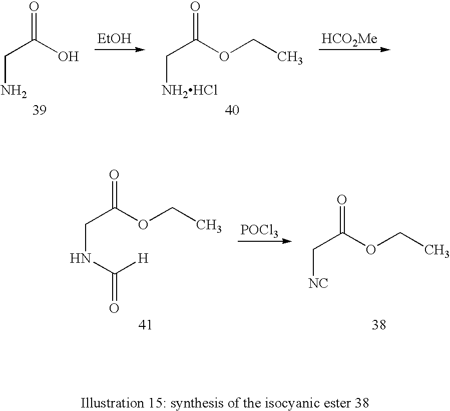

In the synthesis of 38, glycine (39) is to be esterified in the first step with ethanol to yield the glycine ethyl ester (40). This latter compound is to be formylated on the amino function with methyl formate to form the formylamino ester 41. The formylamino function of the resultant 2-formylaminoacetic acid ethyl ester (41) is to be converted into the isocyano function with phosphoryl chloride to form the isocyanoacetic acid ethyl ester (38).

### Example 2

Preparation of the Chiral Auxiliary Reagent: (S,S)-1,2-dimethoxy-1,2-diphenylethane

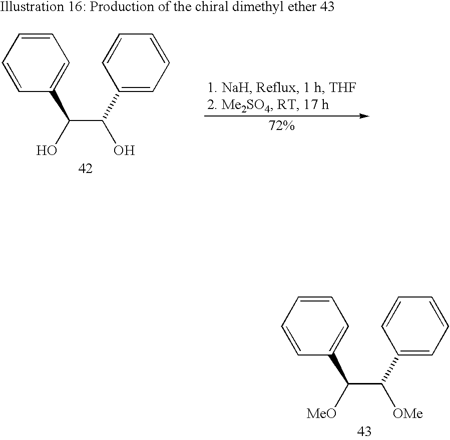

The chiral dimethyl ether 43 was prepared in accordance with a method of K. Tomioka et al, (see Illustration 16)^{[34]}. In this process, purified NaH was initially introduced in excess in THF, (S,S)-hydrobenzoin 42 in THF was added at RT and briefly refluxed. The solution was cooled to 0° C. and dimethyl sulfate was added dropwise. After 30 minutes of stirring, the white, viscous mass was stirred for a further 16 h at RT. After working up and recrystallization from pentane, (S,S)-1,2-dimethoxy-1,2-diphenylethane (43) was obtained in the form of colorless needles and at yields of 72%.

### Example 3

Preparation of Isocyanoacetic Acid Ethyl Ester

The starting compound for synthesis of the isocyanoacetic acid ethyl ester (38) was prepared in accordance with the synthetic pathway shown in Illustration 17:

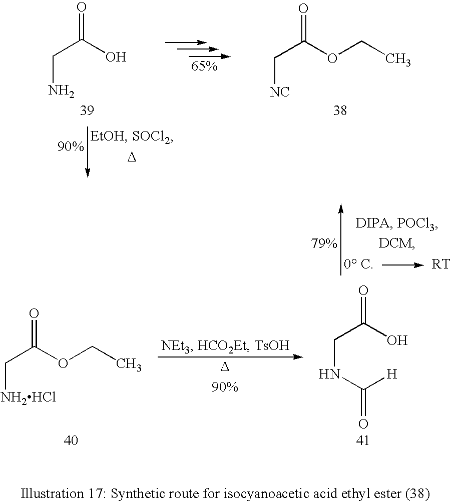

Glycine (39) was here refluxed with thionyl chloride and ethanol, the latter simultaneously acting as solvent, for 2 hours. After removal of excess ethanol and thionyl chloride, the crude ester was left behind as a solid. After recrystallization from ethanol, the glycine ethyl ester was obtained as the hydrochloride (40) in yields of 90-97% in the form of a colorless, acicular solid.

The glycine ethyl ester hydrochloride (40) was formylated on the amino function in accordance with a slightly modified synthesis after C.-H. Wong et al.^{[35]}. The glycine ester hydrochloride 40 was here suspended in methyl formate and toluenesulfonic acid was added thereto in catalytic quantities. The mixture was refluxed. Triethylamine was then added dropwise and refluxing of the reaction mixture was continued. Once the reaction mixture had cooled, the precipitated ammonium chloride salt was filtered out. Any remaining ethyl formate and triethylamine were stripped out from the filtrate and the crude ester was obtained as an orange oil. After distillation, the 2-formylaminoacetic acid ethyl ester (41) was obtained as a colorless liquid in yields of 73-90%.

The formylamino group was converted into the isocyano group in accordance with a method of I. Ugi et al.^{[36]}. The formylaminoacetic acid ethyl ester (41) was introduced into diisopropylamine and dichloromethane and combined with phosphoryl chloride with cooling. Once addition was complete, the temperature was raised to RT and the reaction mixture was then hydrolyzed with 20% sodium hydrogen carbonate solution. After working up and distillative purification, the isocyanoacetic acid ethyl ester (38) was obtained in yields of 73-79% as a light yellow, photosensitive oil.

Using phosphoryl chloride made it possible to avoid the handling difficulties associated with phosgene. In so doing in this stage, a reduction in yield of approx. 10% according to the literature^{[37],[38]}was accepted.

An overall yield of 65% was achieved over three stages, it being straightforwardly possible to perform the first two stages in large batches of up to two moles. In contrast, due to the large quantity of solvent and the elevated reactivity of phosphoryl chloride, the final stage could only be performed in smaller batches of up to 0.5 mol.

### Example 4

Preparation of (E)- and (Z)-2-formylamino-3-methyl-2-octenoic Acid Ethyl Ester

The (E)- and (Z)-2-formylamino-3-methyl-2-octenoic acid ethyl esters (34) were prepared in accordance with a method after U. Schollkopf et al.^{[39],[40]}. The isocyanoacetic acid ethyl ester (38) was deprotonated in a position in situ at low temperatures with potassium tert.-butanolate. A solution of 2-heptanone (37) in THF was then added dropwise. After 30 minutes' stirring, the temperature was raised to room temperature. The reaction was terminated by the addition of equivalent quantities of glacial acetic acid.

The 2-formylamino-3-methyl-2-octenoic acid ethyl ester (34) was still in the form of (EIZ) mixtures, wherein these could readily be separated by chromatography. The overall yields of the purified and separated (E) and (Z) isomers amounted to 73% in the form of colorless solids.

In this reaction, which Schöllkopf^{[41]} termed “formylaminomethylenation of carbonyl compounds”, the oxygen of the ketone is replaced by the (formylamino-alkoxycarbonyl-methylene) group and the β-substituted c-formylaminoacrylic acid ester 34 is directly formed in a single operation. According to Schollkopf, the reaction is based on the mechanism shown in Illustration 18^{[42]}.

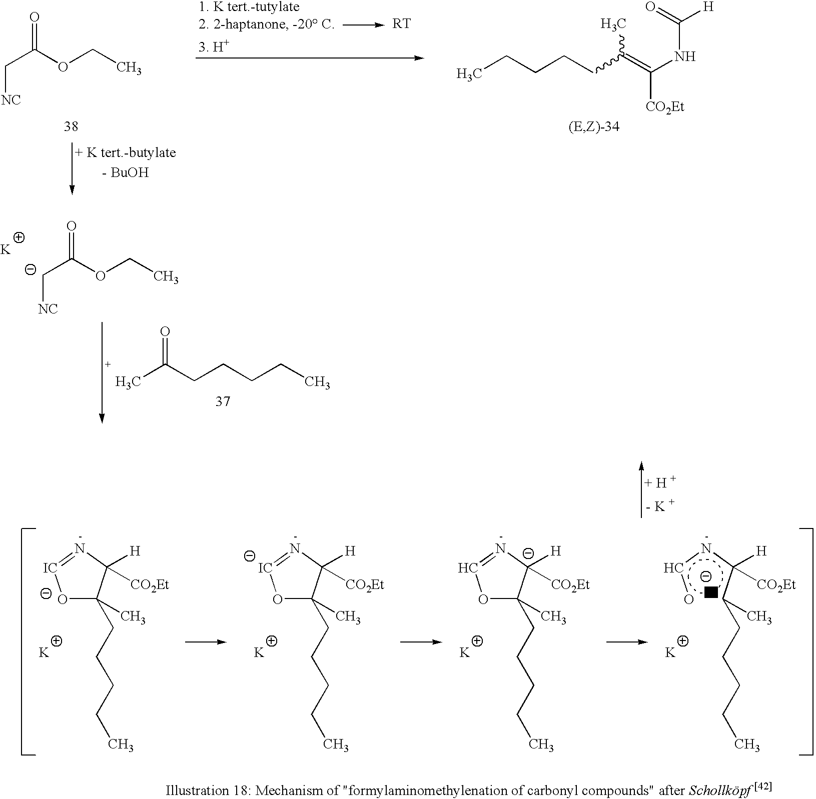

In this reaction, the isocyanoacetic acid ethyl ester 38 is first deprotonated in the a position with potassium tert.-butylate. The carbanion then subjects the carbonyl C atom on the ketone 37 to nucleophilic attack. After several intramolecular rearrangements of the negative charge and subsequent protonation, the substituted a-formylaminoacrylic acid esters 34 are obtained.

Since the 2-formylamino-3-methyl-2-octenoic acid ethyl esters (34) are always obtained in (E/Z) mixtures, the question arose of the possible influence of temperature on the (E/Z) ratio.

TABLE 4
Influence of reaction temperature on the (E/Z) ratio.

| Reaction temperature | (E/Z) ratio^{[a]} |
|---|---|
| 0° C. → RT | 57:43 |
| −40° C. → RT | 63:37 |
| −78° C. → RT | 62:38 |

^{[a]}determined by ¹³C-NMR

Table 4 shows the influence of temperature on (EIZ) ratios. The reactions were performed under the above-described conditions. Only the initial temperatures were varied.

It can be seen that temperature had only a slight influence on the (E/Z) ratios. However, since both isomers are required for the synthesis, the balanced ratio at approx. 0° C. is advantageous since both isomers could be obtained in approximately equal quantities by chromatography.

(E/Z) assignment was carried out after U. Schöllkopf^{[39]}, in accordance with which the protons of the methyl group in P position of the (Z) isomer absorb at a higher field than do those of the (E) isomer^{[43]}.

### Example 5

Michael Addition with Thiols as Donor

A) Tests with Thiolates as Catalyst

Since the Michael addition of thiols onto 2-formylamino-3-methyl-2-octenoic acid ethyl ester (i) does not proceed without a catalyst, a method after T Naito et al.^{[44]} was initially used. In this method, a mixture of thiol and lithium thiolate was first produced in a 10:1 ratio, before the 2-formylaminoacrylic acid ethyl ester 34 was added.

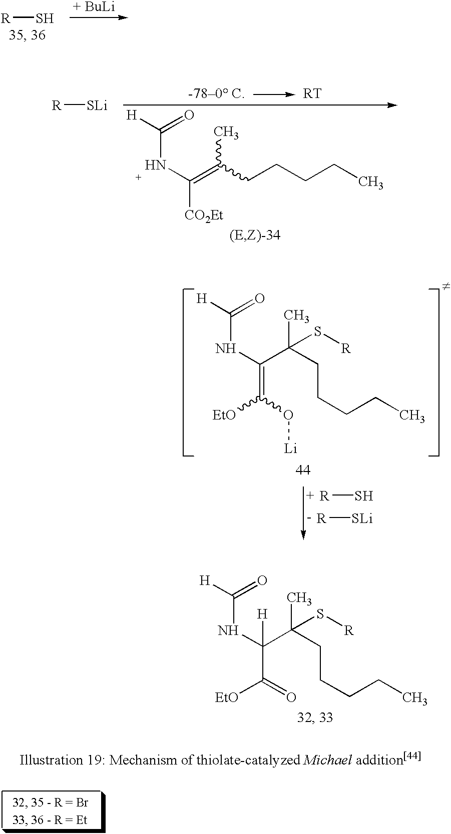

The reaction is assumed to be based on the mechanism shown in Illustration 19^{[44]}. After addition of the thiolates 35 or 36 onto the 2-formylamino-3-methyl-2-octenoic acid ethyl ester [(E,Z)-34] in P position, this adduct 44 is directly protonated by the thiol, which is present in excess, so forming the Michael adduct 32, 33.

The Michael adducts 32, 33 were prepared by initially introducing 0.1 equivalents of BuLi in THF and adding 10 equivalents of thiol at 0° C. The (E) or (Z)-34 dissolved in THF was then added dropwise at low temperature and the mixture was slowly raised to RT.

After hydrolysis with 5% NaOH and column chromatography, 32, 33 were obtained as colorless, viscous oils, in the form of diastereomer mixtures.

Table 5 lists the Michael adducts prepared in accordance with the described synthesis:

TABLE 5
Prepared Michael adducts.

| Educt | Thiol | T [° C.] | Product | dr^{[a]} | de [%]^{[a]} | Yield |
|---|---|---|---|---|---|---|
| (Z)-34 | 35 | −78° C. → RT | 32 | 58:42 | 16 | 83% |
| (Z)-34 | 35 | −25° C. → −15° C. | 32 | 59:41 | 18 | 98% |
| (E)-34 | 35 | −78° C. → RT | 32 | 41:59 | 18 | 79% |
| (Z)-34 | 36 | −78° C. → RT | 33 | 57:43 | 14 | 82% |

^{[a]}determined by ^{13 C-NMR after chromatography}

As can be seen from Table 5, while selection of the formylamino-3-methyl-2-octenoic acid ethyl ester does predetermine (Z)-34 or (E)-34, only the preferential diastereoisomer was determined as a consequence. It was not possible in THF to achieve better predetermination with de values of >18%, as the reaction only starts in this medium at >-20° C. and better control is not to be anticipated at higher temperatures.

The threo/erythro diastereomers 32 could initially be separated from one another by preparative HPLC. As a result, it was found that the threo diastereomer (threo)-32 was a solid, while the erythro diastereomer (erythro)-32 was a viscous liquid.

The attempt was thus made to separate the threo/erythro diastereomers 32 from one another by crystallization. The diastereomer mixtures 32 were dissolved in the smallest possible quantities of pentane/ethanol (˜10:1) and cooled to −22° C. for a period of at least 5 d, during which the diastereomer (threo)-32 crystallized out as a solid. In this manner the enriched diastereomers (threo)-32 and (erythro)-32 were obtained with diastereomeric excesses of 85-96% for (threo)-32 and of 62-83% for (erythro)-32.

B) Tests with Lewis Acids as Catalyst

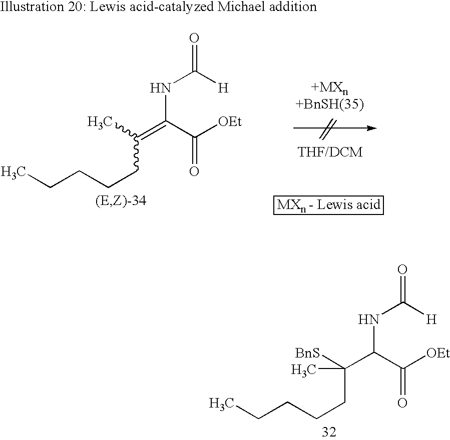

As can be seen in Illustration 20, the attempt was made to catalyze the Michael addition of benzyl mercaptan onto 2-formylaminoacrylic acid ethyl ester 34 by adding a Lewis acid MXₙ. There are many examples of the activation of α,β-unsaturated esters by various Lewis acids for the addition of thiols^{[27]}. In this case, one of the postulated complexes A or B would be formed in which the metal is coordinated on the carbonyl oxygen (see Illustration 21).

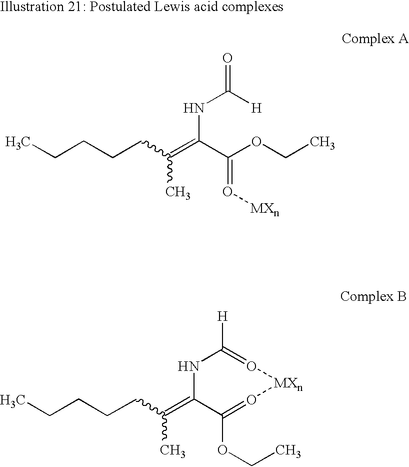

The double bond should be so strongly activated by this complex that the reaction proceeds directly.

The Lewis acids MXₙ listed in Table 6 were tested in various solvents for their catalytic action on this Michael reaction. In these tests, one equivalent of the 2-formylamino-3-methyl-2-octenoic acid ethyl ester (34) were initially introduced in THF or DCM and one equivalent of the dissolved or suspended Lewis acid was added at 0° C. 1.2 equivalents of benzyl mercaptan were then added dropwise and the mixture raised to room temperature after 2 h. Some of the batches were also refluxed, if there was no discernible reaction after one day.

TABLE 6
Tested Lewis acids for catalysis of Michael addition.

| Lewis acid | | Temperature | |
|---|---|---|---|
| MXₙ | Solvent | T | Conversion^{[a]} |
| TiCl₄ | DCM | RT | no conversion after 18 h |
| Ti(O-i-Pr)₃Cl | THF | RT | no conversion after 18 h |
| YbTf₃ | DCM | RT | no conversion after 3 d |
| YbTf₃ | THF | 1 d RT + 1 d | no conversion after 2 d |
| | | reflux | |
| YCl₃ | DCM | RT | no conversion after 3 d |
| SnTf₂ | DCM | RT | no conversion after 3 d |
| ZnTf₂ | DCM | RT | no conversion after 3 d |
| ZnCl₂ | THF | RT | no conversion after 4 d |
| SnCl₄ | DCM | 1 d RT + 1 d | no conversion after 2 d |
| | | reflux | |
| SnCl₄ | THF | 1 d RT + 1 d | no conversion after 2 d |
| | | reflux | |
| BF₃.EtO₂ | DCM | RT | no conversion after 2 d |
| AlCl₃ | THF | RT | no conversion after 2 d |

^{[a]}determined by TLC samples or by NMR

Only with TiCl₄ was there a color change, which would indicate formation of a complex. In contrast, there was no color change indicating the formation of a complex with any of the other Lewis acids. None of the tested Lewis acids exhibited any catalytic action, as there was no identifiable conversion in any of the cases after a reaction time of up to 3 days and the educts could be recovered in their entirety.

C) Testing of Catalysis with Lewis Acids with the Addition of Bases

The Michael addition of thiols onto α,β-unsaturated ketones may be catalyzed as described in section 1.2.4 by the addition of bases (for example triethylamine)^{[45]}. The Bronsted base here increases the nucleophilic properties of the thiol to such a level that it is capable of initiating the reaction.

When reacting equimolar quantities of 2-formylamino-3-methyl-2-octenoic acid ethyl ester (34), benzyl mercaptan (35) and triethylamine in THF, no catalytic action could be observed at reaction temperatures of up to 60° C. The starting materials could be recovered.

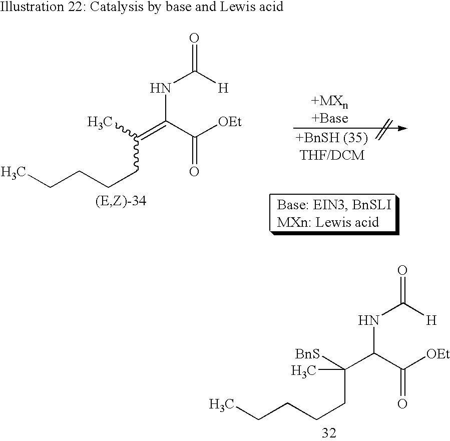

The idea of combining Lewis acid catalysis (presented in section 2.6.2) with base catalysis (see Illustration 22), thus arose because catalysis did not work with Lewis acids or Bronsted bases alone.

In the combinations of bases and Lewis acids shown in Table 7, one equivalent of 2-formylamino-3-methyl-2-octenoic acid ethyl ester (L) was initially introduced in the stated solvent and a solution prepared from 1.2 equivalents of benzyl mercaptan (35) and 1 equivalent of the stated base was added dropwise at 0° C. After 2 h the mixture was raised to room temperature and stirred for a further 3 days. There was no discernible conversion with any of the combinations of bases and Lewis acids. Even in the batch in which benzyllithium thiolate was used as the base in combination with TiCl₄, there was no observable conversion, although without the addition of TiCl₄ complete conversion could be achieved even at 0° C.

TABLE 7
Tested combinations of bases and
Lewis acids for catalysis of Michael addition.

| Lewis acid | Base | Solvent | Conversion^{[a]} |
|---|---|---|---|
| — | NEt₃ | THF | − |
| TiCl₄ | NEt₃ | THF | − |
| TiCl₄ | BnSLi | THF | − |
| TiCl₄ | BnSLi | THF | + |
| TiCl₄ | NEt₃ | DCM | − |
| AlCl₃ | NEt₃ | THF | − |

^{[a]}determined by TLC samples

D) Influence of the Solvent

The question then arose of identifying the suitable solvent in order possibly to achieve higher de values under reaction conditions as described in section 2.6.1 by varying the solvent.

TABLE 8
Influence of solvent on the addition of
benzyl mercaptan (35) onto (E,Z)-34

| | | | Reaction | | |
|---|---|---|---|---|---|
| Educt | Solvent | Temperature | time | dr^{[a]} | de [%]^{[a]} |
| (Z)-34 | THF | −20° C. → −15° C. | 2 h | 59:41 | 18 |
| (E)-34 | THF | −78° C. → RT | 2 h | 41:59 | 18 |
| (Z)-34 | Ether | −25° C. → 5° C. | 2 h | 63:27 | 26 |
| (Z)-34 | Toluene | 0° C. → RT | 18 h | 72:28 | 44 |
| (E)-34 | Toluene | 0° C. → RT | 18 h | 32:68 | 36 |
| (Z)-34 | DCM | 0° C. → RT | 7 d-17 d^{[b]} | 75:25 | 50 |
| (E)-34 | DCM | 0° C. → RT | 7 d-17 d^{[b]} | 25:75 | 50 |

^{[a]}determined by ¹³C-NMR after chromatography
^{[b]}only approx. 50% conversion

As can be seen from Table 8, the de value could be raised by selecting other solvents. A distinct rise was evident with the nonpolar solvents such as toluene and DCM. In this case, de values of 50% were achieved, but the reaction time increased from 2 h in THF to 17 d in DCM. Moreover, with DCM, conversion of only 50% was observable after 7-17 d.

E) Tests of Control by Complexation of the Michael Donor

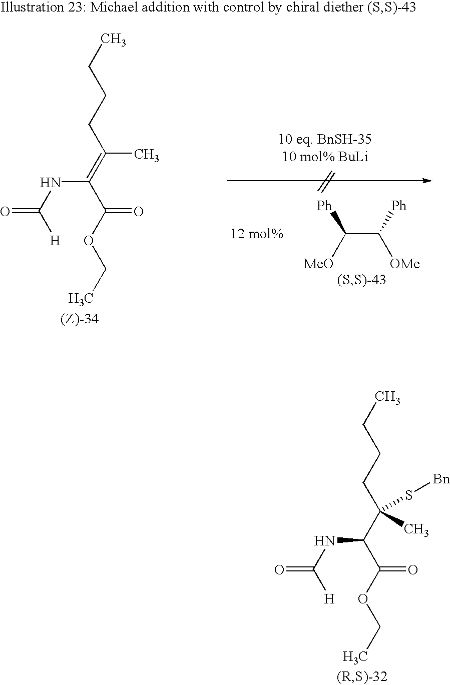

The aim was to control the Michael reaction by the addition of a chiral compound to the thiolate-catalyzed reaction (see section 2.6.1) (see Illustration 23).

Control was achieved according to Tomioka et al.^{[33]} by chiral bi- or triethers. The benzyllithium thiolate was used in this case in only catalytic quantities. Addition of the chiral dimethyl ether (S,S)-43 was intended to complex the lithium thiolate, in order to control the attack thereof. Instead of the diastereomer mixture produced according to sections 2.5.1 and 2.5.4, the intention was to form only one diastereomer enantioselectively.

It is assumed that the chelate shown in Illustration 24 is formed^{[32]}. In this chelate, the lithium thiolate is complexed by both the oxygen atoms of the dimethyl ether. On attack, the carbonyl oxygen of the Michael acceptor 34 also coordinates on the central lithium atom, so controlling the reaction.

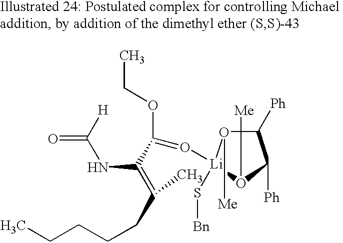

TABLE 9
Tests of control with the chiral dimethyl ether (S,S)-43.

| | | Chiral | | | |
|---|---|---|---|---|---|
| | | diether (S,S)- | Reaction | | ee [%]^{[b]}of the |
| Educt | Solvent | 43 | time | dr^{[a]} | diastereomers |
| (Z)-34 | THF | — | 2 h | 59:41 | 0 |
| (Z)-34 | Ether | 0.12 eq | 2 h | 63:37 | 5-7 |
| (Z)-34 | Toluene | 0.12 eq | 18 h | 71:29 | 4 |
| (Z)-34 | Toluene | — | 18 h | 72:28 | 1-4 |
| (Z)-34 | DCM | 0.12 eq | 17 d | 75:25 | 1-9 |
| (Z)-34 | DCM | — | 17 d | 79:21 | 414 6 |
| (E)-34 | Toluene | 0.12 eq | 18 h | 30:70 | 1 |
| (E)-34 | Toluene | — | 18 h | 32:68 | 0 |
| (E)-34 | DCM | 0.12 eq | 7 d | 25:75 | 5-7 |
| (E)-34 | DCM | · | 7 d | 32:68 | 1-6 |
| (E)-34 | THF | — | 2 h | 41:59 | 0 |

^{[a]}by ¹³C-NMR spectroscopy after chromatography
^{[b]}according to HPLC_{anal.}

Testing of control by the dimethyl ether (S,S)-43 was performed in ether, DCM and toluene. 0.1 equivalents of BuLi were initially introduced at 0° C. and 10 equivalents of benzyl mercaptan 35 were added. 0.12 equivalents of the dissolved dimethyl ether (S,S)-43 were added thereto. However, no color change indicating the formation of a complex was to be seen. 30 min later, one equivalent of 2-formylamino-3-methyl-2-octenoic acid ethyl ester 34 was added dropwise at 0° C. The reaction was terminated after the time stated in each case by the addition of 5% NaOH. The diastereomeric excesses were determined by chromatography from the ¹³C-NMR spectra after purification by column spectroscopy. The enantiomeric excesses were determined after crystallization of the diastereomers (threo)-32 (pentane/ethanol) by analytical HPLC on a chiral stationary phase.

As can be seen from Table 9, no chiral induction of the Michael addition was discernible from the addition of the chiral dimethyl ether, as the measured enantiomeric excesses are within the accuracy of the HPLC method. The reason for this is that the purified diastereomers are contaminated with the other diastereomer and it was not possible to measure all four isomers together with baseline separation.

### Example 6

Summary

In the context of the present invention, a synthetic route was first of all devised for the preparation of (E,Z)-2-formylaminoacrylic acid esters (E,Z)-34. This was achieved with a four stage synthesis starting from glycine (L9). After esterification, N-formylation, condensation of the N-formylamino function and olefination (E,Z)-34 was obtained in an overall yield of 47% and with an (E/Z)-ratio of 1:1.3 (see Illustration 25).

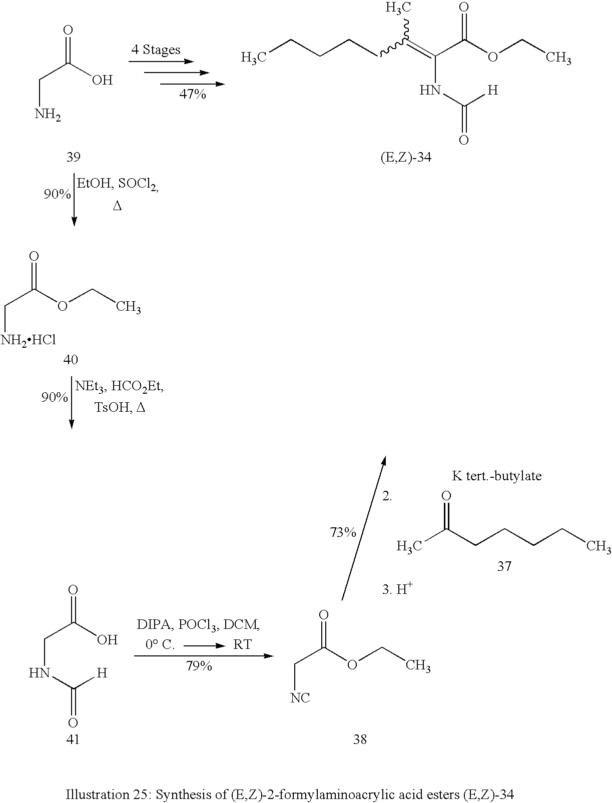

It was intended to add mercaptans onto the synthesized (E,Z)-2-formylaminoacrylic acid esters (E,Z)-34 in a Michael addition. The reaction could be catalyzed by addition of 0.1 equivalents of lithium thiolate.

In order to enable enantioselective control by means of chiral catalysts, the use of various catalysts was investigated, which may subsequently be provided with chiral ligands. Lewis acids, Bronsted bases and a combination of the two were tested in various solvents for their catalytic action (see Illustration 26). However, no catalytic systems have yet been found for the desired Michael addition.

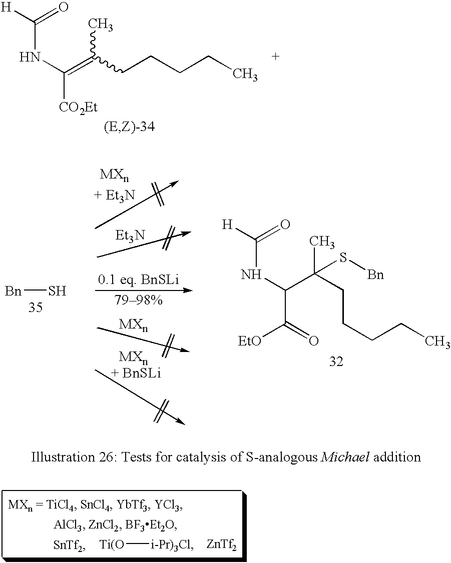

A mixture of both diastereomers was obtained from thiolate-catalyzed Michael addition. By changing solvent, the diastereomeric excess when using (Z)-34 could be raised from 17% (THF) to 43% (toluene) and 50% (DCM). Starting from (E)-34, comparable de values were achieved with the inverse diastereomeric ratio. However, as the de value increases, so too does the reaction time from 2 h (THF) to up to 17 d (DCM), in order to achieve satisfactory conversion.

By crystallising the threo diastereomer (threo)-32 from pentane/ethanol (10:1), the threo and erythro diastereomers 32 could be further purified to a de value of 96% for (threo)-32 and 83% for (erythro)-32.

On the basis of the successful catalysis with 0.1 equivalents of thiolate, the attempt was made to control the attack of thiolate by addition of the chiral diether (S,S)-1,2-dimethoxy-1,2-diphenylethane [(S,S)-43]^{[33]}. Nonpolar solvents were used for this purpose. However no influence of the diether (S,S)-43 on the control of the reaction has yet been observable.

### Example 7

Use of TMSCl

Since the diastereomer separation developed in the present invention works well, the thiolate may be used stoichiometrically as shown in Example 5A and the adduct preferably scavenged with TMSCl as the enol ether 45. Protonating this adduct 45 with a chiral proton donor R*-H makes it possible to control the second center (see Illustration 27).

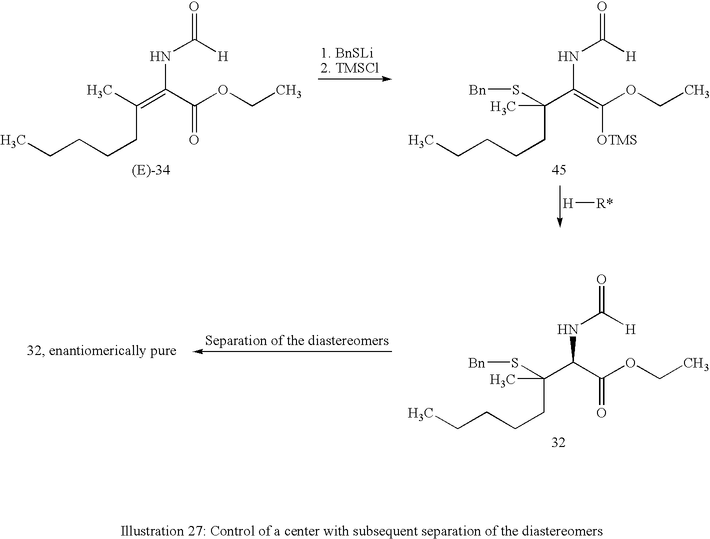

The two enantiomerically pure diastereomers formed may, as described, be separated by crystallization. This type of control makes all four stereoisomers individually accessible.

### Example 8

Use of Sterically Demanding Groups:

A second possibility for controlling Michael addition is intramolecular control by sterically demanding groups, preferably the TBDMS group. These may be introduced enantioselectively using a method of D. Enders and B. Lohray^{[46],[47]}. The α-silyl ketone 47 produced starting from acetone (6) was then reacted with isocyanoacetic acid ethyl ester (38) to yield the 2-formylamino-3-methyl-4-(t-butyldimethylsilyl)-2-octenoic acid ethyl ester (E)-48 and (Z)-48 (see Illustration 28).

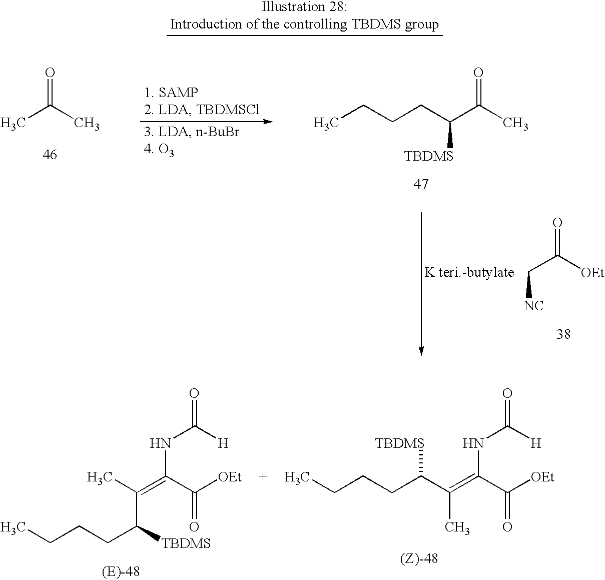

(E)-48 and (Z)-48 are then reacted with a thiol in a Michael addition, wherein the reaction is controlled by the TBDMS group and the (E/Z) isomers. The controlling TBDMS group may be removed again by the method of T Otten^{[12]} with n-BuNF4/NH₄F/HF as the elimination reagent, the publication of T. Otten^{[12]} being part of the disclosure. This is another possibility for synthesizing all four stereoisomers mutually independently.

Since the initially presented, alternative synthesis offers the possibility of asymmetric catalysis on protonation of the silyl enol ether 45, this route is the better alternative. The second alternative route may possibly also suffer the problem of silyl group elimination, as the N-formyl group may sometimes also be eliminated under the elimination conditions to form the hydrofluoride.

### Example 8

Experimental Conditions: Comments on Preparative Operations

A) Protective Gas Method

All air- and moisture-sensitive reactions were performed under an argon atmosphere in evacuated, heat treated flasks sealed with septa.

Liquid components or components dissolved in solvent were added using plastic syringes fitted with V2A hollow needles. Solids were introduced through a countercurrent stream of argon.

B) Solvents

Solvent absolution was carried out on predried and prepurified solvents:

| | |
|---|---|
| Tetrahydro- | Four hours' refluxing over calcium hydride followed by |
| furan: | distillation. |
| Abs. | Two hours' refluxing of pretreated THF over sodium- |
| tetrahydro- | lead alloy under argon followed by distillation. |
| furan: | |
| Dichloro- | Four hours' refluxing over calcium hydride followed by |
| methane: | distillation through a 1 m packed column. |
| Abs. | Shaking of the pretreated dichloromethane with conc. |
| dichloro- | sulfuric acid, neutralisation, drying, two hours' refluxing |
| methane: | over calcium hydride under argon followed by |
| | distillation. |
| Pentane: | Two hours' refluxing over calcium hydride followed by |
| | distillation through a 1 m packed column. |
| Diethyl ether: | Two hours' refluxing over KOH followed by distillation |
| | through a 1 m packed column. |
| Abs. diethyl | Two hours' refluxing over sodium-lead alloy under |
| ether: | argon followed by distillation. |
| Toluene: | Two hours' refluxing over sodium wire followed by |
| | distillation through a 0.5 m packed column. |
| Abs. toluene: | Two hours' refluxing over sodium-lead alloy followed |
| | by distillation. |
| Methanol: | Two hours' refluxing over magnesium/magnesium |
| | methanolate followed by distillation. |

C) Reagents Used

| | |
|---|---|
| Argon: | Argon was purchased from Linde. |
| n-Butyllithium: | n-BuLi was obtained as a 1.6 molar solution |
| | in hexane from Merck. |
| (S,S)-(−)-1,2-diphenyl- | was purchased from Aldrich. |
| 1,2-ethanediol: | |
| Benzyl mercaptan: | was purchased from Aldrich |
| Ethyl mercaptan: | was purchased from Fluka. |
| 2-Heptanone: | was purchased from Fluka. |

All remaining reagents are also commercially available and were purchased from companies such as Aldrich, Fluka, Merck and Acros.

D) Reaction Monitoring

Thin-layer chromatography was used for reaction monitoring and for detection after column chromatography (see section 3.1.5). TLC was performed on silica gel coated glass sheets with a fluorescence indicator (Merck, silica gel 60, 0.25 mm layer). Detection was achieved by fluorescence quenching (absorption of UV light of a wavelength of 254 nm) and by dipping in Mostain reagent [5% solution of (NH₄)₆Mo₇O₂₄ in 10% sulfuric acid (v/v) with addition of 0.3% Ce(SO₄)₂] followed by heating in a stream of hot air.

E) Product Purification

The substances were mainly purified by column chromatography in glass columns with an integral glass frit and silica gel 60 (Merck, grain size 0.040-0.063 mm). An overpressure of 0.1-0.3 bar was applied. The eluents were generally selected such that the R_{f} value of the substance to be isolated was 0.35. The composition of the solvent mixtures was measured volumetrically. The diameter and length of the column was tailored to the separation problem and the quantity of substance.

Some crystalline substances were also purified by recrystallization in suitable solvents or mixtures.

F) Analysis

| | |
|---|---|
| HPLCₚᵣₑₚₐᵣₐₜᵢᵥₑ | Gilson Abimed; column: Hibar ® ready-to-use column |
| | (25 cm × 25 mm) from Merck and UV detector. |
| HPLC_{analytical:} | Hewlett Packard, column: Daicel OD, UV detector |
| ¹H-NMR | Varian GEMINI 300 (300 MHz) and Varian Inova 400 |
| spectroscopy: | (400 MHz) with tetramethylsilane as internal standard. |
| ¹³C-NMR | Varian GEMINI 300 (75 MHz) and Inova 400 (100 |
| spectroscopy. | MHz) with tetramethylsilane as internal standard. |
| 2D-NMR | Varian Inova 400. |
| spectroscopy: | |
| Gas | Siemens Sichromat 2 and 3; FID detector, columns: |
| chromato- | OV-17-CB (fused silica, 25 m × 0.25 mm ID); CP-Sil-8 |
| graphy: | (fused silica, 30 m × 0.25 mm ID). |
| IR | a) Measurements of KBr pellets: Perkin-Elmer FT/IR |
| spectroscopy: | 1750. |
| | b) Measurements in solution: Perkin-Elmer FT/IR 1720 |
| | X. |
| Mass | Varian MAT 212 (EL 70 eV, CL 100 eV). |
| spectroscopy: | |
| Elemental | Heraeus CHN-O-Rapid, Elementar Vario EL. |
| analysis: | |
| Melting points: | Tottoli melting point apparatus, Büchi 535. |

G) Comments on Analytical Data

| Yields: | The stated yields relate to the isolated, purified products |
|---|---|
| Boiling | The stated boiling temperatures were measured inside |
| point/pressure: | the apparatus with mercury thermometers and are |
| | uncorrected. The associated pressures were measured |
| | with analogous sensors. |
| ¹H-NMR | The chemical shifts δ are stated in ppm against |
| spectroscopy: | tetramethylsilane as internal standard, and the coupling |
| | constants J are stated in hertz (Hz). The following |
| | abbreviations are used to describe signal multiplicity: s = |
| | singlet, d = doublet, t = triplet, q = quartet, |
| | q = quintet, m = multiplet. cz denotes a complex zone |
| | of a spectrum. A prefixed br indicates a broad signal. |
| ¹³C-NMR | The chemical shifts δ are stated in ppm with |
| spectroscopy: | tetramethylsilane as internal standard. |
| de values: | Diastereomeric excesses (de) are determined with the |
| | assistance of the ¹³C-NMR-spectra of the compounds. |
| | This method exploits the different shifts of |
| | diastereomeric compounds in the proton-decoupled ¹³C |
| | spectrum. |
| IR | The position of the absorption bands (\tilde{v}) is stated in |
| spectroscopy: | cm⁻¹. The following abbreviations are used to |
| | characterise the bands: vs = very strong, s = strong, |
| | m = moderate, w = weak, vw = very weak, br = broad. |
| Gas | The retention time of the undecomposed compounds is |
| chromato- | stated in minutes. Details of measurement conditions |
| graphy: | are then listed: colunm used, starting temperature, |
| | temperature gradient, final temperature (in each case in |
| | ° C.) and the injection temperature Tₛ, if different from |
| | the standard temperature. (Sil 8: Tₛ = 270° C., OV-17: |
| | Tₛ = 280° C.) |
| Mass | The masses of the fragment ions (m/z) are stated as a |
| spectroscopy: | dimensionless number, the intensity of which is a |
| | percentage of the base peak (rel. intensity). High |
| | intensity signals (>5%) or characteristic signals are |
| | stated. |
| Elemental | Values are stated as mass percentages [%] of the stated |
| analysis: | elements. The samples were deemed authentic at Δ_{C,H,N} |
| | ≦ 0.5%. |

### Example 10

General Procedures (GP)

Preparation of Glycine Alkyl Ester Hydrochlorides [GP 1]

1.2 equivalents of thionyl chloride are introduced into 0.6 ml of alcohol per mmol of glycine with ice cooling to −10° C. After removal of the ice bath, 1 equivalent of glycine is added in portions. The mixture is stirred for 2 hours while being refluxed. After cooling to room temperature, the excess alcohol and the thionyl chloride are removed in a rotary evaporator. The resultant white solid is combined twice with the alcohol and the latter is again removed in the rotary evaporator in order to remove any adhering thionyl chloride completely.

Preparation of Formylaminoacetic Acid Alkyl Esters [GP 2]

1 equivalent of glycine alkyl ester hydrochloride is suspended in 0.8 ml of ethyl or methyl formate per mmol of glycine alkyl ester hydrochloride. 130 mg of toluenesulfonic acid are added per mol of glycine alkyl ester hydrochloride and the mixture is refluxed. 1.1 equivalents of triethylamine are now added dropwise to the boiling solution and the reaction solution is stirred overnight while being refluxed.

After cooling to RT, the precipitated ammonium chloride salt is filtered out, the filtrate is evaporated to approx. 20% of its original volume and cooled to −5° C. The reprecipitated ammonium chloride salt is filtered out, the filtrate evaporated and distilled at 1 mbar.

Preparation of Isocyanoacetic Acid Alkyl Ester [GP 3]

1 equivalent of formylaminoacetic acid alkyl ester and 2.7 equivalents of diisopropylamine are introduced into DCM (10 ml per mmol formylaminoacetic acid alkyl ester) and cooled to −3° C. with an ice bath. 1.2 equivalents of phosphoryl chloride are then added dropwise and the mixture is then stirred for a further hour at this temperature. Once the ice bath has been removed and room temperature reached, the mixture is cautiously hydrolyzed with 1 ml of 20% sodium carbonate solution per mmol of formylaminoacetic acid alkyl ester. After approx. 20 min, vigorous foaming is observed and the flask has to be cooled with ice water. After 60 minutes' stirring at RT, further water (1 ml per mmol of formylaminoacetic acid alkyl ester) and DCM (0.5 ml per mmol formylaminoacetic acid alkyl ester) are added. The phases are separated and the organic phase is washed twice with 5% Na₂CO₃ solution and dried over MgSO₄. The solvent is removed in a rotary evaporator and the remaining brown oil is distilled.

Preparation of (E)- and (Z)-2-formylamino-3-dialkyl-2-propenoic Acid Alkyl Esters [GP4]

1.05 equivalents of potassium tert.-butanol in 0.7 ml of THF per mmol of isocyanoacetic acid alkyl ester are cooled to −78° C. To this end, a solution prepared from 1.0 equivalent of isocyanoacetic acid alkyl ester in 0.25 ml of THF per mmol is slowly added and the mixture is stirred at this temperature for 30 min (pink-colored suspension). A solution of 1.0 equivalent of ketone in 0.125 ml of THF per mmol is now added dropwise. After 30 minutes' stirring at −78° C., the temperature is raised to RT (1 h) and 1.05 equivalents of glacial acetic acid are added in a single portion (yellow solution) and the mixture is stirred for a further 20 minutes. The solvent is removed in a rotary evaporator (40° C. bath temperature). The crude product is obtained as a solid. The solid is suspended in 1.5 ml of diethyl ether per mmol and 0.5 ml water is added per equivalent. The clear phases are separated and the aqueous phase extracted twice with diethyl ether. The combined organic phases are washed with saturated NaHCO₃ solution and dried over MgSO₄. After removal of the solvent, a waxy solid is obtained. The (E) and (Z) products can be separated by chromatography with diethyl ether/pentane (4:1) as eluent.

Preparation of 2-formylamino-3-dialkyl-3-alkylsulfanylpropanoic Acid Alkyl Ester [GP5]

0.1 equivalents of butyllithium are introduced into 50 ml of THF per mmol and are cooled to 0° C. 10 equivalents of the mercaptan are now added dropwise. After 20 minutes' stirring, the solution is cooled to a temperature between −40 and 0° C. and 1 equivalent of the 2-formylamino-3-dialkyl-2-propenoic acid alkyl ester in 5 ml of THF per mmol is slowly added. The mixture is stirred at the established temperature for 2 h and the temperature is then raised to 0° C. and the mixture hydrolyzed with 5% sodium hydroxide solution. The phases are separated and the aqueous phase is extracted twice with DCM. The combined organic phases are dried over MgSO₄ and the solvent is removed in a rotary evaporator. The mercaptan, which was introduced in excess, may be separated by means of chromatography with DCM/diethyl ether (6:1) as eluent.

### Example 11

Special Procedures and Analytical Data

A) (S,S)-(−)-1,2-dimethoxy-1,2-diphenylethane ((SS)-43)

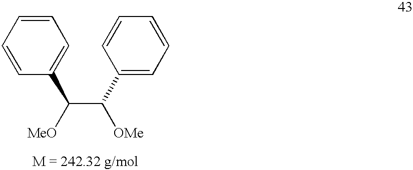

140 mg of NaH (60% in paraffin) are washed three times with pentane and dried under vacuum. The resultant material is then suspended in 5 ml of abs. THF. 250 mg (1.17 mmol) of (S,S)-(−)-2,2-diphenyl-2,2-ethanediol (42) dissolved in 3 ml of THF are now added dropwise. After the addition, the mixture is stirred for 30 minutes while being refluxed and is then cooled to 5° C. 310 mg of dimethyl sulfate are slowly added dropwise and the mixture is stirred for a further 30 min with ice cooling. The ice bath is removed and the reaction mixture raised to RT, wherein a viscous white solid is obtained which is stirred overnight at RT. The reaction is terminated by the addition of 5 ml of saturated NH₄Cl solution. The phases are separated and the aqueous phase is extracted twice with diethyl ether. The combined organic phases are washed first with saturated NaHCO₃ solution and then with brine and dried over MgSO₄. After removal of the solvent in a rotary evaporator, a colorless solid is obtained which is recrystallized in pentane (at −22° C.). The dimethyl ether is now obtained in the form of colorless needles.

| | | |
|---|---|---|
| Yield: | 204 mg | (0.84 mmol, 72% of theory) |
| mp: | 98.5° C. | (Lit.: 99-100° C.)^{[39]} |
| GC: | Rₜ = 3.08 min | (OV-17, 160-10-260) |

¹H-NMR spectrum (400 MHz, CDCl₃):

δ=7.15 (m, 6H, Hr), 7.00 (m, 4H, Her), 4.31 (s, 2H, CHOCH₃), 3.27 (s, 6H, CH₃) ppm.

¹³C-NMR spectrum (100 MHz, CDCl₃):

δ=138.40 (C_{Ar, quart}.), 128.06 (4×HC_{Ar}), 127.06 (HC_{Ar, para}), 87.98 (CH₃), 57.47 (HCOCH₃) ppm.

IR Spectrum (KBr Pellet):

\tilde{v}=3448 (br m), 3082 (vw), 3062 (m), 3030 (s), 2972 (s), 2927 (vs), 2873 (s), 2822 (vs), 2583 (vw), 2370 (vw), 2179 (vw), 2073 (vw), 1969 (br m), 1883 (m), 1815 (m), 1760 (w), 1737 (vw), 1721 (vw), 1703 (w), 1686 (vw), 1675 (vw), 1656 (w), 1638 (vw), 1603 (m), 1585 (w), 1561 (w), 1545 (w), 1525 (vw), 1492 (s), 1452 (vs), 1349 (s), 1308 (m), 1275 (w), 1257 (vw), 1215 (vs), 1181 (m), 1154 (m), 1114 (vs), 1096 (vs), 1028 (m), 988 (s), 964 (s), 914 (m), 838 (s), 768 (vs), 701 (vs), 642 (m), 628 (s), 594 (vs), 515 (s) [cm⁻¹].

Mass Spectrum (Cl, isobutane):

M/z [%]=212 (M⁺+1−OMe, 16), 211, (M⁺−MeOH, 100), 165 (M⁺−Ph, 2), 121 (½ M⁺, 15), 91 (Bn⁺, 3), 85 (M⁺−157, 8), 81 (M⁺−161, 7), 79 (M⁺−163, 6), 71 (M⁺−171, 8).

Elemental analysis:

| | | |
|---|---|---|
| calc.: | C = 79.31 | H = 7.49 |
| fd.: | C = 79.12 | H = 7.41 |

All other analytical data are in line with literature values^{[34]}.

B) Glycine Ethyl Ester Hydrochloride (40)

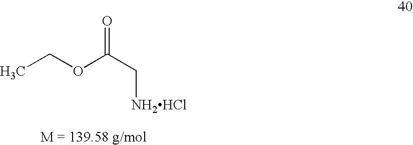

In accordance with GP 1, 1000 ml of ethanol are reacted with 130 g (1.732 mol) of glycine 39 and 247.3 g (2.08 mol) of thionyl chloride. After recrystallization from ethanol, a colorless, acicular solid is obtained, which is dried under a high vacuum.

| | | |
|---|---|---|
| Yield: | 218.6 g | (1.565 mol, 90.4% of theory) |
| GC: | Rₜ = 1.93 min | (OV-17, 60-10-260) |
| mp.: | 145° C. | (Lit.: 144° C.)^{[48]} |

¹H-NMR spectrum (300 MHz, CD₃OD):

δ=4.30 (q, J=7.14, 2H, OCH₂), 3.83 (s, 2H, H₂CNH₂), 1.32 (tr, J=7.14, 3H, CH₃) ppm.

¹³C-NMR spectrum (75 MHz, CD₃OD):

δ=167.53 (C═O), 63.46 (OCH₂), 41.09 (H₂CNH₂), 14.39 (CH₃) ppm.

All other analytical data are in line with literature values

C) N-formyl Glycine Ethyl Ester (4)

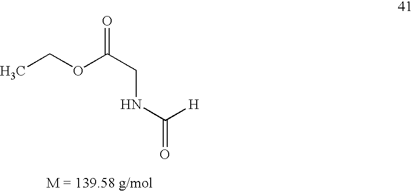

In accordance with GP 2, 218 g (1.553 mol) of glycine ethyl ester hydrochloride 40, 223 mg of toluenesulfonic acid and 178 g of triethylamine are reacted in 1.341 of ethyl formate. After distillation at 1 mbar, a colorless liquid is obtained.

| | | |
|---|---|---|
| Yield: | 184.0 g | (1.403 mol, 90.3% of theory) |
| GC: | Rₜ = 6.95 min | (CP-Sil 8, 60-10-300) |
| bp.: | 117° C./1 mbar | (Lit.: 119-120° C./1 mbar)^{[49]} |

A rotameric ratio of 94:6 around the N—CHO bond is obtained.

¹H-NMR spectrum (400 MHz, CDCl₃):

δ=8.25, 8.04 (s, d, J=11.81, 0.94H10.06H. HC═O), 4.22 (dq, J=7.14, 3.05, 2H, OCH₂), 4.07 (d,J=5.50, 2H_{1—}CC═O), 1.29 (tr,J=7.14, 3H, CH₃) ppm.

¹³C-NMR spectrum (100 MHz, CDCl₃):

δ=169.40 (OC═O), 161.43 (HC═O), 61.55 (OCH₂), 39.90 (H₂CNH₂), 14.10 (CH₃) ppm.

All other analytical data are in line with literature values^{[49]}.

D) Isocyanoacetic Acid Ethyl Ester (38)

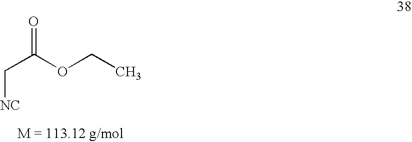

In accordance with GP 3, 50 g (381 mmol) of formyl glycine ethyl ester 41, 104 g (1.028 mol) of diisopropylamine and 70.1 g (457 mmol) of phosphoryl chloride are reacted in 400 ml of DCM. After distillation at 5 mbar a slightly yellow liquid is obtained.

| | | |
|---|---|---|
| Yield: | 34.16 g | (302 mmol, 79.3% of theory) |
| GC: | Rₜ = 1.93 min | (OV-17, 50-10-260) |
| bp.: | 77° C./5 mbar | (Lit.: 89-91° C./20 mbar)^{[50]} |

¹H-NMR spectrum (300 MHz, CDCl₃):

δ=4.29 (q, J=7.14, 2H, OCH₂), 4.24 (d, J=5.50, 2H, H₂CC═O), 1.33 (tr, J=7.14, 3H, CH₃) ppm.

¹³C-NMR spectrum (75 MHz, CDCl₃):

δ=163.75 (OC═O), 160.87 (NC), 62.72 (OCH₂), 43.58 (H₂CNH₂), 14.04 (CH₃) ppm.

IR-spectrum (Capillary):

\tilde{v}=2986 (s), 2943 (w), 2426 (br vw), 2164 (vs, NC), 1759 (vs, C═O), 1469 (w), 1447 (w), 1424 (m), 1396 (vw), 1375 (s), 1350 (s), 1277 (br m), 1213 (vs), 1098 (m), 1032 (vs), 994 (m), 937 (vw), 855 (m), 789 (br m), 722 (vw), 580 (m), 559 (w) [cm¹].

Mass Spectrum (Cl, Isobutane):

M/z [%]=171 (M⁺+isobutane, 6), 170 (M⁺+isobutane−1, 58), 114 (M⁺+1, 100), 113 (M⁺, 1), 100 (M⁺−13, 2), 98 (M⁺−CH₃, 2), 87 (M⁺−C₂H₅+1, 1), 86 (M⁺−C₂H₅, 18), 84 (M⁺−29, 2).

All other analytical data are in line with literature values^{[50]}.

E) (E)- and (Z)-2-formylamino-3-methyl-2-octenoic Acid Ethyl Ester ((E,Z)-34)

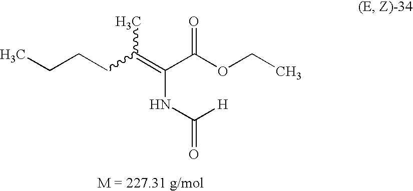

According to GP 4, 15 g (132 mmol) of isocyanoacetic acid ethyl ester 38, 15.6 g (139 mmol) of potassium tert.-butanolate, 15.1 g (132 mmol) of 2-heptanone 37 and 8.35 g (139 mmol) of glacial acetic acid are reacted.

The (E) and (Z) products are separated from one another by chromatography with diethyl ether/pentane (4:1) as eluent:

| | | |
|---|---|---|
| Yield: | 11.52 g | (50.7 mmol, 38.0% of theory) |
| | | (Z) product |
| | 9.07 g | (39.9 mmol, 30.2% of theory) |
| | | (E) product |
| | 1.32 g | (5.8 mmol, 4.4% of theory) |
| | mixed fraction | |

F) (Z)-2-formylamino-3-methyl-2-octenoic Acid Ethyl Ester ((Z)-34)

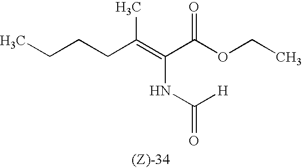

| | | |
|---|---|---|
| GC: | Rₜ = 12.96 min | (CP-Sil 8, 80-10-300) |
| mp.: | 57° C. | (colorless, amorphous) |
| TLC: | R_{f} = 0.32 | (ether:pentane - 4:1) |
| | R_{f} = 0.34 | (DCM:ether - 4:1) |

A rotameric ratio of 65:35 around the N—CHO bond is obtained.

¹H-NMR spectrum (400 MHz, CDCl₃):

δ=8.21, 7.95 (d, d, J=1.38, 11.40, 0.65, 0.35H. HC═O), 6.80, 6.69 (br s, br d, J=11.40, 0.65, 0.35H, HN), 4.22 (dq, J=1.10, 7.14, 2H, OCH₂,), 2.23 (dtr, J=7.97, 38.73, 2H, C═CCH₂), 2.20 (dd, J=1.10, 21.7, 3H, C═CCl₃), 1.45 (dquin, J=1.25, 7.97, 2H, CCH₂CH₂), 1.30 (dquin, J=4.12, 7.14, 4H, CH₃CH₂CH₂), 1.30 (m, 3H, OCH₂CH₃), 0.89 (tr, J=7.00, 3H, CH₂CH₁₃) ppm.

¹³C-NMR spectrum (100 MHz, CDCl₃):

δ=164.82, 164.36 (OC═O), 159.75 (HC═O), 152.72, 150.24 (C═CNH), 120.35, 119.49 (C═CCH₃), 61.11, 60.89 (OCH₂), 35.82, 35.78 (CH₂), 31.80, 31.72 (CH₂), 27.21, 26.67 (CH₂), 22.45, 22.42 (CH₂), 19.53, 19.17 (C═CCH₃), 14.18 (OCH₂CH₃), 13.94, 13.90 (CH₂CH₃) ppm.

IR Spectrum (KBr Pellet):

\tilde{v}=3256 (vs), 2990 (w), 2953 (w), 2923 (m), 2872 (w), 2852 (w), 2181 (br vw), 1711 (vs, C═O), 1659 (vs, OC═O), 1516 (s), 1465 (s), 1381 (s), 1310 (vs), 1296 (vw), 1269 (m), 1241 (s), 1221 (s), 1135 (w), 1115 (vw), 1032 (vs), 1095 (s), 1039 (m), 884 (m), 804 (m), 727 (vw), 706 (vw), 590 (w), 568 (vw) [cm⁻¹].

Mass Spectrum (E1, 70 eV):

M/z [%]=227 (M⁺, 19), 182 (M⁺−EtOH+1, 24), 181 (M⁺−EtOH, 100) 170 (M+-57, 9), 166 (M⁺−61, 8), 156 (M⁺−71, 5), 154 (M⁺−HCO₂Et+1, 6), 153 (M⁺−HCO₂Et, 13), 152(M⁺−HCO₂Et−1, 13), 142 (M⁺−85, 15), 139 (M⁺−HCO₂Et−CH₃+1, 8), 138 (M⁺−HCO₂Et−CH₃, 65), 126 (M⁺−HCO₂Et−CHO+2, 16), 125 (M⁺−HCO₂Et−CHO+1, 34), 124 (M⁺−HCO₂Et−CHO, 51), 114 (M⁺−113, 36), 111 (M⁺−HCO₂Et−HNCHO+1, 17), 110 (M⁺−HCO₂Et−HNCHO, 36), 109 (M⁺−HCO₂Et−HNCHO−1, 20), 108 (M⁺−HCO₂Et−HNCHO−2, 10), 98 (M⁺−129, 6), 97 (M⁺−130, 9), 96 (M⁺−131, 12), 82 (M⁺−145, 10), 68 (M⁺−159, 48), 55 (M⁺−172, 12).

Elemental analysis:

| | | | |
|---|---|---|---|
| calc.: | C = 63.41 | H = 9.31 | N = 6.16 |
| fd.: | C = 63.51 | H = 9.02 | N = 6.15 |

G) (E)-2-formylamino-3-methyl-2-octenoic Acid Ethyl Ester ((E)-34)

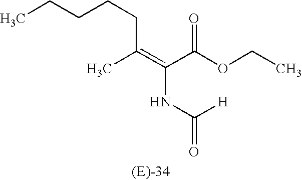

| | | |
|---|---|---|
| GC: | Rₜ = 13.71 min | (CP-Sil 8, 80-10-300) |
| mp.: | 53° C. | (colorless, amorphous) |
| TLC: | R_{f} = 0.20 | (ether:pentane - 4:1) |
| | R_{f} = 0.26 | (DCM:ether - 4:1) |

A rotameric ratio of 65:35 around the N—CHO bond is obtained.

¹H-NMR spectrum (400 MHz, CDCl₃):

δ=8.16, 7.96 (dd, J=1.64, 11.68, 0.65, 0.35H. HC═O), 6.92, 6.83 (br s, br d, J=11.68, 0.65, 0.35H, HN), 4.23 (dq, J=0.82, 7.14, 2H, OCH₂), 2.56 (dtr, J=7.96, 18.13, 2H, C═CCH₂), 1.90 (dd, J=0.55, 39.55, 3H, C═CCH₃), 1.51 (m, 2H, CCH₂CH₂), 1.32 (dquin, J=2.48, 7.14, 4H, CH₃CH₂CH₂), 1.32 (m, 3H, OCH₂CH₁₃), 0.90 (dtr, J=3.57, 7.14, 3H, CH₂CH₃) ppm.

¹³C-NMR spectrum (100 MHz, CDCl₃):

δ=164.75. 164.14 (OC═O), 158.96 (HC═O), 151.38, 150.12 (C═CNH), 120.74, 119.48 (C═CCH₃), 61.10, 60.90 (OCH₂), 35.59 (CH₂), 31.90 (CH₂), 28.09, 28.04 (CH₂), 22.48 (CH₂), 20.89 (C═CCH₃), 14.17 (OCH₂CH₃), 13.99 (CH₂CH₃) ppm.

IR Spectrum (KBr Pellet):

\tilde{v}=3276 (vs), 2985 (w), 2962 (w), 2928 (m), 2859 (m), 2852 (w), 1717 (vs, C═O), 1681 (s, OC═O), 1658 (vs, OC═O), 1508 (s), 1461 (s), 1395 (s), 1368 (vw), 1301 (vs), 1270 (w), 1238 (m), 1214 (s), 1185 (m), 1127 (m), 1095 (s), 1046 (m), 1027 (w), 932 (m), 886 (s), 793 (m), 725 (br s), 645 (m), 607 (m), 463 (w) [cm⁻¹].

Mass Spectrum (E1, 70 eV):

M/z [%]=227 (M⁺, 19), 182 (M⁺−EtOH+1, 20), 181 (M⁺−EtOH, 100), 170 (M⁺−57, 8), 166 (M⁺−61, 8), 156 (M⁺−71, 7), 154 (M⁺−HCO₂Et+1, 6), 153 (M⁺−HCO₂Et, 14), 152 (M⁺−HCO₂Et−1, 12), 142 (M⁺−85, 151), 139 (M⁺−HCO₂Et−CH₃+1, 8), 138 (M⁺−HCO₂Et−CH₃, 58), 126 (M⁺−HCO₂Et−CHO+2, 13), 125 (M⁺−HCO₂Et−CHO+1, 32), 124 (M⁺−HCO₂Et−CHO, 46), 114 (M⁺−113, 31), 111 (M⁺−HCO₂Et−HNCHO+1, 16), 110 (M⁺−HCO₂Et−HNCHO, 34), 109 (M⁺−HCO₂Et−HNCHO−1, 18), 108 (M⁺−HCO₂Et —HNCHO−2, 9), 98 (M⁺−129, 5), 97 (M⁺−130, 7), 96 (M⁺−131, 11), 93 (M⁺−134, 7), 82 (M⁺−145, 9), 69 (M⁺−158, 6), 68 (M⁺−159, 43), 55 (M⁺−172, 10).

Elemental analysis:

| | | | |
|---|---|---|---|
| calc.: | C = 63.41 | H = 9.31 | N = 6.16 |
| fd.: | C = 63.23 | H = 9.38 | N = 6.10 |

H) 3-Benzylsulfanyl-2-formylamino-3-methyloctanoic Acid Ethyl Ester (32)

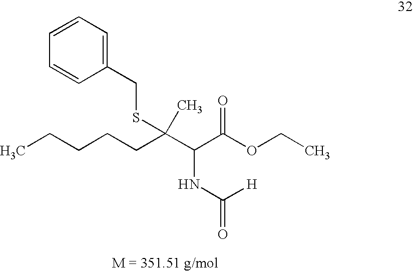

According to GP 5, 0.28 ml (0.44 mmol) of n-butyllithium, 5.5 g (44 mmol) of benzyl mercaptan 35 and 1 g (4.4 mmol) of 2-formylamino-3-methyl-2-octenoic acid ethyl ester (34) are reacted in 40 ml of abs. THF (−78° C. RT). The resultant colorless oil is purified by column chromatography with DCM/ether (6:1), wherein a colorless, high viscosity oil is obtained.

| | | |
|---|---|---|
| Yield: | 1.51 g | (43 mmol, 98% of theory) |
| TLC: | R_{f} = 0.51 | (DCM:ether - 6:1) |

The resultant diastereomers may be separated from one another by preparative HPLC or by crystallization in pentane/ethanol (10:1).

J) threo Diastereomer ((threo)-32):

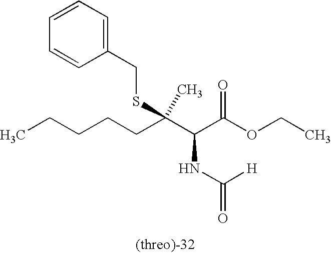

| | | |
|---|---|---|
| mp.: | 75° C. | (colorless, acicular, crystalline) |
| de: | >96% | (according to ¹³C-NMR) |
| HPLC_{prep.}: | 19.38 min | (ether:pentane - 85:15) |

A rotameric ratio of 91:9 around the N—CHO bond is obtained.

¹H-NMR Spectrum (400 MHz, CDCl₃):

δ=8.22, 7.98 (s, d, J=11.54, 0.91, 0.09H, HC═O), 7.21-7.32 (cz, 5H, CHₐᵣ), 6.52, 6.38 (dm, J=8.66, 0.91, 0.09H, HN), 4.74 (d, J=8.66, 1H. C_NH), 4.24 (ddq, J=17.85, 10.71, 7.14, 2H, OCH₂), 3.71 (s, 2H, SCH₂), 1.56 (m, 3H, SCCH₃), 1.45 (dquin, 1.25, 7.97, 2H, CCH₂CH₂), 1.20-1.45 (cz, 11H, CH₃CH₂CH₂CH₂CH₂+OCH₂CH₃), 0.89 (dtr, J=3.3, 7.00, 3H, CH₂CH₃) ppm.

¹³C-NMR Spectrum (100 MHz, CDCl₃):

δ=170.37 (OC═O), 160.90 (HC═O), 137.31 (Cr, quart), 129.31 (H₅C), 128.81 (HC_{Ar}), 127.41 (HC_{Ar, para}), 61.94 (OCH₂), 57.00 (CHNH), 52.30 (CS), 38.59 (CH₂), 33.31 (CH₂), 32.42 (CH₂), 24.00 (CH₂), 22.92 (CH₂), 22.51 (SCCH₃), 14.54 (OCH₂CH₃), 14.42 (CH₂CH₃) ppm.

IR Spectrum (KBr Pellet):

\tilde{v}=3448 (m), 3184 (br vs), 3031 (m), 2975 (m), 2929 (s), 2899 (w), 2862 (m), 1954 (w), 1734 (vs, C═O), 1684 (vs, OC═O), 1601 (w), 1561 (s), 1495 (m), 1468 (s), 1455 (m), 1296 (vw), 1441 (w), 1381 (vs), 1330 (s), 1294 (m), 1248 (s), 1195 (vs), 1158 (w), 1126 (s), 1096 (s), 1070 (w), 1043 (vw), 1028 (w), 1008 (s), 958 (m), 919 (w), 854 (s), 833 (m), 783 (s), 715 (vs), 626 (vw), 626 (m), 567 (vw) 483 (s) [cm⁻¹].

Mass Spectrum (E1, 70 eV):

M/z [%]=351 (M⁺, 1), 324 (M⁺−C₂H₅, 1), 306 (M⁺−C₂H₅OH−1, 1), 278 (M⁺−73, 1), 250 (M⁺−HCO₂Et−HCO, 1), 223 (M⁺−128, 5), 222 (M⁺−129, 16), 221 (M⁺−EtO₂CCHNHCHO, 100), 184 (M⁺−167, 6), 91 (M⁺−260, 71).

Elemental analysis:

| | | | |
|---|---|---|---|
| calc.: | C = 64.92 | H = 8.32 | N = 3.98 |
| fd.: | C = 64.88 | H = 8.40 | N = 3.92 |

K) Erythro Diastereomer ((Erythro)-32):

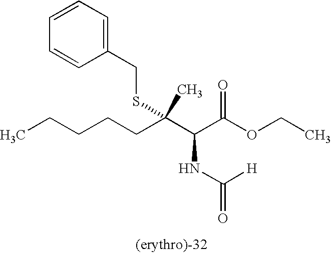

Clear, oily liquid

| | | |
|---|---|---|
| de: | 82% | (according to ¹³C-NMR) |
| HPLC_{prep.}: | 20.61 min | (ether:pentane - 85:15) |

A rotameric ratio of 91:9 around the N—CHO bond is obtained.

¹H-NMR spectrum (400 MHz, CDCl₃):

δ=8.22, 7.97 (s, d, J=11.54, 0.91, 0.09H, _C═O), 7.20-7.34 (cz, 5H, CHₐᵣ), 6.61, 6.43 (br dm, J=9.34, 0.91, 0.09H, _IN), 4.74 (d, J=9.34, 1H, C_NH), 4.24 (ddq, J=17.85, 10.71, 7.14, 2H, OCH₂), 3.77 (d, J=11.53, 1H, SCHH), 3.69 (d,J=11.53, 1H, SCHH), 1.70 (m, 2H, CH₂), 1.52 (m, 2H, CH₂), 1.17-1.40 (cz, 10H, CH₃C+2×CH₂+OCH₂CH₃), 0.90 (tr, J=7.14, 3H, CH₂CH₃) ppm.

¹³C-NMR spectrum (100 MHz, CDCl₃):

δ=169.87 (OC═O), 160.49 (HC═O), 137.05 (C_{Ar, quart}.), 128.91 (HC_{Ar}), 128.40 (HC_{Ar}), 126.99 (HC_{Ar, para}), 61.52 (OCH₂), 56.81 (CHNH), 51.91 (CS), 37.51 (CH₂), 32.83 (CH₂), 32.13 (CH₂), 23.65 (CH₂), 23.19 (CH₂), 22.55 (SCCH₃), 14.11 (OCH₂CH₃), 14.03 (CH₂CH₃) ppm.

IR-Spectrum (Capillary):

\tilde{v}=3303 (br vs), 3085 (vw), 3062 (w), 3029 (m), 2956 (vw), 2935 (vw), 2870 (w), 2748 (w), 1949 (br w), 1880 (br w), 1739 (vs, C═O), 1681 (vs, OC═O), 1603 (m), 1585 (vw), 1496 (br vs), 1455 (vs), 1381 (br vs), 1333 (s), 1197 (br vs), 1128 (w), 1095 (m), 1070 (s), 1030 (vs), 971 (br w), 918 (m), 859 (s), 805 (vw), 778 (m), 714 (vs), 699 (vw), 621 (w), 569 (w) 484 (s) [cm¹].

Mass Spectrum (E1, 70 eV):

M/z [%]=351 (M⁺, 1), 324 (M⁺−C₂H₅, 1), 306 (M⁺−C₂H₅OH−1, 1), 278 (M⁺−73, 1), 250 (M⁺−HCO₂Et —HCO, 1), 223 (M⁺−128, 6), 222 (M⁺−129, 17), 221 (M⁺−EtO₂CCHNHCHO, 100), 184 (M⁺−167, 6), 91 (M⁺−260, 70).

Elemental analysis:

| | | | |
|---|---|---|---|
| calc.: | C = 64.92 | H = 8.32 | N = 3.98 |
| fd.: | C = 64.50 | H = 8.12 | N = 4.24 |

L) 3-Ethylsulfanyl-2-formylamino-3-methyloctanoic Acid Ethyl Ester (3)

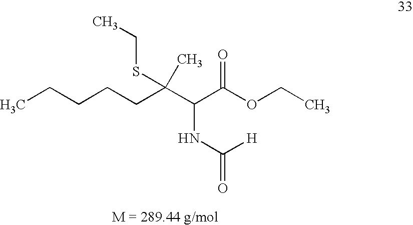

According to GP 5, 0.28 ml (0.44 mmol) of n-butyllithium, 2.73 g (44 mmol) of ethyl mercaptan 36 and 1 g (4.4 mmol) of (E)-2-formylamino-3-methyl-2-octenoic acid ethyl ester (E)-34 are reacted in 40 ml of abs. THF (−78° C.→RT). A colorless oil is obtained, which is purified by column chromatography with DCM/ether (6:1). The product is obtained as a colorless, viscous oil.

| | | |
|---|---|---|
| Yield: | 1.05 g | (36.3 mmol, 82% of theory) |
| de: | 14% | (according to ¹H— and ¹³C—NMR) |
| TLC: | Rf = 0.49 | (DCM:ether - 4:1) |

A rotameric ratio of 91:9 around the N—CHO bond is obtained.

¹H-NMR spectrum (400 MHz, CDCl₃, diastereomer mixture):

δ=8.26 (s, 0.91H, _C═O), 8.02 (d, J=11.82+d, J 11.81, 0.09H. HC═O), 6.79 (d, J=9.34+d, J=8.71, 0.91H, HN), 6.55 (m, 0.09H, HN), 4.77 (d, J=9.34, 0.57H, CHNH), 4.64 (d, J=8.71, 0.43H. CHNH), 4.22 (m, 2H, OCH₂), 2.50 (m, 2H, SCH₂), 1.43-1.73 (cz, 4H, 2×CH₂), 1.20-1.37 (cz, 10H), 1.18 (tr, J=7.42+tr, J=7.00, 3H, SCH₂CH₃), 0.90 (dtr, J=4.71, 7.14, 3H, CH₂CH₃) ppm.

¹³C-NMR spectrum (100 MHz, CDCl₃, diastereomer mixture):

δ=170.36, 170.25 (OC═O), 160.98, 160.93 (HC═O), 61.74, 61.70 (OCH₂), 57.15, 57.02 (CHNH), 51.19 (SC_{quart}), 38.66, 37.86 (CH₂), 32.51, 32.42 (CH₂), 23.94 (CH₂), 23.45, 22.50 (SCCH₃), 22.90, 22.85 (CH₂), 22.17, 22.11 (CH₂), 14.44, 14.41 (OCH₂H₃), 14.38, 14.36 (SCH₂CH₃), 14.27, 14.25 (CH₂CH₃) ppm.

IR-Spectrum (Capillary):

\tilde{v}=3310 (br s), 2959 (s), 2933 (vs), 2871 (s), 2929 (s), 2746 (br w), 1739 (vs, C═O), 1670 (vs, OC═O), 1513 (br s), 1460 (m), 1468 (m), 1381 (s), 1333 (m), 1298 (vw), 1262 (w), 1196 (vs), 1164 (vw), 1127 (m), 1096 (m), 1070 (w), 1030 (s), 978 (w), 860 (m), 833 (m), 727 (br m) [cm⁻¹].

Mass Spectrum (E1, 70 eV):

M/z [%]=289 (M⁺, 1), 260 (M⁺−C₂H₅, 1), 244 (M⁺−C₂H₅OH−1, 1), 228 (M⁺−SC₂H₅, 1), 188 (M⁺−HCO₂Et−HCO, 1), 161 (M⁺−128, 5), 160 (M⁺−129, 11), 159 (M⁺−EtO₂CCHNHCHO, 100), 97 (M⁺−192, 11), 89 (M⁺−200, 11), 75 (M⁺−214, 5), 55 (M⁺−214, 14).

Elemental analysis:

| | | | |
|---|---|---|---|
| calc.: | C = 58.10 | H = 9.40 | N = 4.84 |
| fd.: | C = 57.97 | H = 9.74 | N = 5.13 |

The threo diastereoisomer (threo)-33 could be obtained in elevated purity by 30 days' crystallization in pentane/ethanol:

M) Threo Diastereomer ((threo)-33):

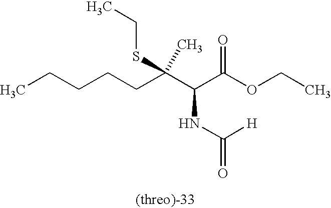

| | | |
|---|---|---|
| de: | 86% | (according to ¹³C-NMR) |
| mp: | 45.5° C. | (colorless, crystalline) |

A rotameric ratio of 91:9 around the N—CHO bond is obtained.

¹H-NMR spectrum (300 MHz, CDCl₃):

δ=8.26, 8.01 (br s, dd, J=11.81H, 0.91, 0.09H. HC═O), 6.61, 6.40 (dm, J=9.06, 0.91, 0.09H, _N), 4.77 (d, J=9.34, 0.57H, CHNH), 4.22 (ddq, J=7.14, 10.72, 17.79, 2H, OCHR), 2.50 (ddq, J=7.42, 10.72, 27.36, 2H, SCHE), 1.42-1.76 (cz, 4H, 2×CH₂), 1.24-1.38 (cz, 10H), 1.18 (dtr, J=3.3, 7.42, 3H, SCH₂CH₃), 0.90 (tr, J=7.14, 3H, CH₂CH₃) ppm.

¹³C-NMR spectrum (75 MHz, CDCl₃):

δ=170.13 (OC═O), 160.71 (HC═O), 61.50 (OCH₂), 56.85 (CHNH), 50.97 (SC_{quart}.), 37.64 (CH₂), 32.22 (CH₂), 23.66 (CH₂), 23.47 (SCCH₃), 22.60 (CH₂), 21.81 (CH₂), 14.09 (OCH₂CH₃), 14.07 (SCH₂CH₃), 13.93 (CH₂CH₃) ppm.

IR Spectrum (KBr Pellet):

\tilde{v}=3455 (m), 3289 (br s), 3036 (w), 2981 (s), 2933 (vs), 2860 (vs), 2829 (s), 2755 (br m), 2398 (vw), 2344 (vw), 2236 (vw), 2062 (w), 1737 (vs, C═O), 1662 (vs, OC═O), 1535 (s), 1450 (m), 1385 (s), 1373 (s), 1334 (vs), 1267 (m), 1201 (vs), 1154 (m), 1132 (s), 1118 (w), 1065 (m), 1050 (w), 1028 (s), 1016 (m), 978 (m), 959 (vw), 929 (w), 896 (m), 881 (m), 839 (w), 806 (m), 791 (m), 724 (s), 660 (m), 565 (m) [cm⁻¹].

Mass Spectrum (Cl, Isobutane):

M/z [%]=346 (M⁺+isobutane−1, 2), 292 (M⁺+3, 6), 291 (M⁺+2, 17), 290 (M⁺+1, 100), 245 (M⁺−C₂H₅OH, 1), 228 (M⁺−SC₂H₅, 6), 159 (M⁺−EtO₂CCHNHCHO, 8).

Elemental analysis:

| | | | |
|---|---|---|---|
| calc.: | C = 58.10 | H = 9.40 | N = 4.84 |
| fd.: | C = 58.05 | H = 9.73 | N = 4.76 |

It has hitherto been possible to obtain diastereoisomer (erythro)-33 only with a de of 50% by crystallization of (threo)-33; no separate analysis was performed for this.

List of Abbreviations

List of Abbreviations

| | |
|---|---|
| GP | general procedure |
| abs. | absolute |
| eq. | equivalent |
| AcCl | acetyl chloride |
| Ar | aromatic |
| calc. | calculated |
| Bn | benzyl |
| Brine | saturated NaCl solution |
| BuLi | butyllithium |
| TLC | thin-layer chromatography |
| DIPA | diisopropylamine |
| DCM | dichloromethane |
| de | diastereomeric excess |
| DMSO | dimethyl sulfoxide |
| dr | diastereomeric ratio |
| ee | enantiomeric excess |
| Et | ethyl |
| et al. | et altera |
| GC | gas chromatography |
| fd. | found |
| sat. | saturated |
| HPLC | high pressure liquid chromatography |
| IR | infrared |
| conc. | concentrated |
| Lit. | literature reference |
| Me | methyl |
| min | minute |
| MS | mass spectroscopy |
| NMR | nuclear magnetic resonance |
| quart. | quaternary |
| Pr | propyl |
| R | organic residue |
| RT | room temperature |
| bp. | boiling point |
| mp. | melting point |
| TBS | tert.-butyldimethylsilyl |
| Tf | triflate |
| THF | tetrahydrofuran |
| TMS | trimethylsilyl |
| TsOH | toluenesulfonic acid |
| v | volume |

### LIST OF LITERATURE REFERENCES

1. D. Enders, R. W. Hoffmanm, Ch. i. u. Z. 1985, 19, 177.

2. L. Pasteur, Ann. Chim. 1848, 24, 442.

3. J. A. Le Bel, Bull Soc. Chim. Fr. 1874, 22, 337.

4. J. H. van't Hoff, Bull. Soc. Chim. Fr. 1875, 23, 295.

5. T. Laue, A. Plagens, Namen-und Schlagwort-Reaktionen der Organischen Chemie, B. G. Teubner Verlag Stuttgart 1998.

6. R. Bruckner, Reaktionsmechanismen, Spektrum Akademischer Verlag Heidelberg 1996.

7. E. E. Bergmann, D. Ginsburg, R. Rappo, Org. React. 1959, 10, 179.

8. T. Hudlicky, J. D. Price, Chem. Rev. 1989, 89, 1467.

9. H. Scherer, Dissertation, RWTH Aachen, 1991.

10. S. G. Pyne, P. Bloem, S. L. Chapman, C. E. Dixon, R. Griffith, J. Org. Chem 1990, 55, 1086.

11. E. S. Gubnitskaya, L. P. Peresypkina, L. I. Samarai, Russ. Chem. Rev. 1990, 59, 807.

12. T. Otten, Dissertation, RWTH Aachen, 2000.

13. D. Enders, H. Wahl, W. Bettray, Angew. Chem. 1995, 107, 527.

14. V. S. Martin, M. T. Ramirez, M. S. Soler, Tetrahedron Lett. 1990, 31, 763.

15. W. Amberg, D. Seebach, Chem. Ber. 1990, 123, 2250.

16. D. Enders, K. Heider, G. Raabe, Angew. Chem. 1993, 105, 592.

17. A. Kamimura, H. Sasatani, T. Hashimoto, T. Kawai, K. Hori, N. Ono, J. Org. Chem. 1990, 55, 3437.

18. W. D. Rudorf, R. Schwarz, Wiss. Z.-Martin-Luther Univ. Halle-Wittenberg, Math. Naturwiss. Reihe 1989, 38, 25.

19. K. Tomioka, A. Muraoka, M. Kanai, J. Org. Chem. 1995, 60, 6188.

20. T. Naito, O. Miyata, T. Shinada, I. Ninomiya, Tetrahedron 1997, 53, 2421.

21. a) D. A. Evans, D. M. Ennis, D. J. Mathre, J. Am. Chem. Soc. 1982, 104, 1737.

b) D. A. Evans, K. T. Chapman, J. Bisaha, J. Am. Chem. Soc. 1988, 110, 1238.

22. A. A. Schleppnik, F. B. Zienty, J. Org. Chem. 1964, 39, 1910.

23. T. Mukaiyama, K. Suzuki, I. Ikegawa, Bull. Chem. Soc. Jpn. 1982, 55, 3277.

24. CD Römpp Chemie Lexikon-Version 1.0, Stuttgart/New York: Georg Thieme Verlag 1995.

25. A. Kumar, R. V. Salunkhe, R. A. Rane, S. Y. Dike, J. Chem. Soc., Chem. Commun. 1991, 485.

26. H. Wynberg, H. Hiemstra, J. Am. Chem. Soc. 1981, 103, 417.

27. T. Mukaiyama, T. Izawa, K. Saigo, H. Takei, Chem. Lett. 1973, 355.

28. D. A. Evans, M. C. Willis, J. N. Johnston, Org. Lett. 1999, 1, 865.

29. S. Kanemasa, Y. Oderaotoshi, E. Wada, J. Am. Chem. Soc. 1999, 121, 8675.

30. B. L. Feringa, E. Keller, N. Veldman, A. L. Speck, Tetrahedron Asymmetry 1997, 8, 3403.

31. M. Shibasaki, T. Arai, H. Sasai, J. Am. Chem. Soc. 1998, 120, 4043.

32. K. Tomioka, Synthesis 1990, 541.

33. K. Tomioka, K. Nishimura, M. Ono, Y. Nagaoka, J. Am. Chem. Soc. 1997, 119, 12974.

34. K. Tomioka, M. Shindo, K. Koga, J. Org. Chem. 1998, 63, 9351.

35. C. H. Wong, W. K. C. Park, M. Auer, H. Jasche, J. Am. Chem. Soc. 1996, 118, 10150.

36. I. Ugi, R. Obrecht, R. Herrmann, Synthesis 1985, 400.

37. I. Ugi, U. Fetzer, U. Eholzer, H. Knupper, K. Offermann, Angew. Chem. 1965, 11, 492.

38. I. Maeda, K. Togo, R. Yoshida, Bull. Chem. Soc. Jpn. 1971, 44, 1407.

₃₉. U. Schöllkopf, R. Meyer, Liebigs Ann. Chem. 1981, 1469.

40. U. Schöllkopf, R. Meyer, Liebigs Ann. Chem. 1977, 1174.

41. U. Schöllkopf, F. Gerhart, R. Schroder, Angew. Chem. 1969, 87, 701.

42. U. Schöllkopf, F. Gerhart, R. Schroder, D. Hoppe, Liebigs Ann. Chem. 1972, 766, 1174.

43. R. K. Olsen, A. Srinivasan, K. D. Richards, Tetrahedron Lett. 1976, 12, 891.

44. T. Naito, O. Miyata, T. Shinada, I. Ninomiya, T. Date, K. Okamura, S. Inagaki, J. Org. Chem. 1991, 56, 6556.

45. D. N. Reinhoudt, V. van Axel Castelli, A. Dalla Cort, L Mandolini, L. Schiaffino, Chem. Eur. J. 2000, 6, 1193.

46. D. Enders, B. B. Lohray, Angew. Chem. 1987, 99, 360.

47. D. Enders, B. B. Lohray, Angew. Chem. 1988, 100, 594.

48. Beilstein 4, 1, 342.

49. R. G. Jones, J. Am. Chem. Soc. 1949, 71, 644.

50. I. Maeda, K. Togo, R. Yoshida, Bull. Chem. Soc. Jpn. 1971, 44, 1407.

What is claimed is:

1. A process for producing a compound of formula 31 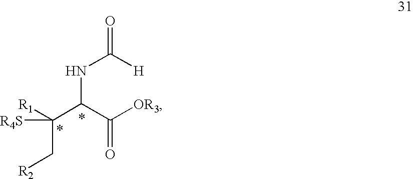
in which
R¹, R² and R³ are independent y of one another a C₁₋₁₀ alkyl, saturated or unsaturated, branched or unbranched, mono- or polysubstituted or unsubstituted;
* indicates a stereoselective center,
R⁴ is C₁₋₁₀ alkyl, saturated or unsaturated, branched or unbranched, mono- or polysubstituted or unsubstituted; C₃₋₈ cycloalkyl, saturated or unsaturated, unsubstituted or mono- or polysubstituted; aryl or heteroaryl, in each case unsubstituted or mono- or polysubstituted; or aryl, C₃₋₈ cycloalkyl or heteroaryl, in each case unsubstituted or mono- or polysubstituted, attached via saturated or unsaturated C₁₋₃ alkyl;
the process comprising
reacting a compound of formula 30, under Michael addition conditions with a compound of the formula R₄SH, in accordance with reaction I below

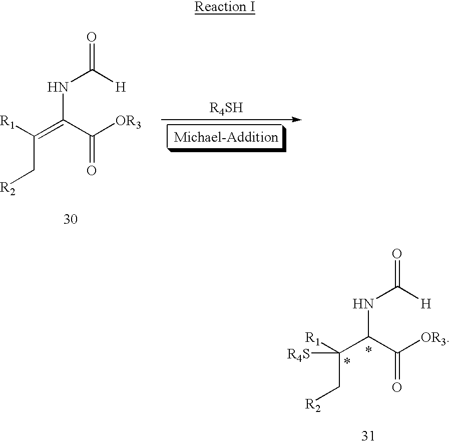

2. A process according to claim 1, wherein in Reaction I a chiral catalyst is used, wherein the chiral catalyst is selected from the group consisting of a chiral auxiliary reagent, a Lewis acid and a Brønsted base, or a combination thereof.

3. A process according to claim 2, wherein the chiral auxiliary reagent is diether (S, S)-1,2-dimethoxy-1,2-diphenylethane.

4. A process according to claim 1, wherein the compound of formula 31 is hydrolyzed with a base.

5 A process according to claim 4, wherein the base is NaOH.

6. A process according to claim 1, wherein the compound of formula 31 is further purified.

7. A process according to claim 6, wherein the compound of formula 31 is purified by column chromatography.

8. A process according to claim 1, wherein the compound R₄SH is used as a lithium thiolate or is converted into lithium thiolate during or before reaction I.

9. A process according to claim 8, wherein butyllithium (BuLi) is used before reaction I to convert the compound of the formula R₄SH into lithium thiolate.

10. A process according to claim 9, wherein an equivalent ratio of BuLi:R₄SH of between 1:5 and 1:20 is used.

11. A process according to claim 9, wherein the equivalent ratio of BuLi:R₄SH is 1:10.

12. A process according to claim 1, wherein at the beginning of reaction I, the reaction temperature is not higher than 0° C., and over the course of reaction I, the temperature is adjusted to room temperature.

13. A process according to claim 1, wherein at the beginning of reaction I, the reaction temperature is between about −70 and about −80° C.

14. A process according to claim 1, wherein at the beginning of reaction I, the reaction temperature is about −78° C.

15. A process according to claim 1, wherein at the beginning of reaction I, the reaction temperature is not higher than 0° C., and over the course of reaction I, the temperature is adjusted to between about −20° C. and about −10° C.

16. A process according to claim 15, wherein at the beginning of reaction I, the reaction temperature is at between about −30 and about −20° C., and over the course of reaction I, the temperature is adjusted to between about −20° C. and about −10° C.

17. A process according to claim 16, wherein at the beginning of reaction I, the reaction temperature is at about −25° C., and over the course of reaction I, the temperature is adjusted to −15° C.

18. A process according to claim 1, wherein reaction I proceeds in an organic solvent.

19. A process according to claim 1, wherein reaction I proceeds in a nonpolar solvent.

20. A process according to claim 1, wherein the organic solvent is toluene, ether, tetrahydrofuran (THF) or dichloromethane (DCM).

21. A process according to claim 1, wherein diastereomers of the compound of formula 31 are separated after reaction I.

22. A process according to claim 21, wherein diastereomers are separated by preparative HPLC or crystallization.

23. A process according to claim 22, wherein diastereomers are separated by crystallization using pentane/ethanol (10:1) as solvent and cooling.

24. A process according to claim 21, wherein enantiomers of the compound of formula 31 are separated prior to the separation of the diastereomers.

25. A process according to claim 1, wherein R1 is C₁₋₆ alkyl, saturated or unsaturated, branched or unbranched, mono- or polysubstituted or unsubstituted; and R² is C₂₋₉ alkyl, saturated or unsaturated, branched or unbranched, mono- or polysubstituted or unsubstituted.

26. A process according to claim 25, wherein R¹ is C₁₋₂ alkyl, mono- or polysubstituted or unsubstituted, and R² is C₂₋₉ alkyl, saturated or unsaturated, branched or unbranched, mono- or polysubstituted or unsubstituted.

27. A process according to claim 25, wherein R¹ is methyl or ethyl.

28. A process according to claim 25, wherein R² is C₂₋₇ alkyl.

29. A process according to claim 25, wherein R² is ethyl, propyl, n-propyl, i-propyl, butyl, n-butyl, i-butyl, tert.-butyl, pentyl, hexyl or heptyl.

30. A process according to claim 1, wherein R¹ is methyl and R2 is n-butyl.

31. A process according to claim 1, wherein R³ is C₁₋₃ alkyl, saturated or unsaturated, branched or unbranched, mono- or polysubstituted or unsubstituted.

32. A process according to claim 31, wherein R³ is methyl or ethyl.

33. A process according to claim 1, wherein R⁴ is C₁₋₆ alkyl, saturated or unsaturated, branched or unbranched, mono- or polysubstituted or unsubstituted; phenyl or thiophenyl, unsubstituted or monosubstituted; or unsubstituted or monosubstituted phenyl attached via CH₃.

34. A process according to claim 33, wherein R⁴ is phenyl or thiophenyl monosubstituted with OCH₃, CH₃, OH, SH, CF₃, F, Cl, Br or I.

35. A process according to claim 33, wherein R⁴ is phenyl monosubstituted with OCH₃, CH₃, OH, SH, CF₃, F, Cl, Br or I, attached via CH₃.

36. A process according to claim 33, wherein R⁴ is saturated, unbranched and unsubstituted, C₁₋₆ alkyl.

37. A process according to claim 34, wherein R⁴ is methyl, ethyl, propyl, n-propyl, i-propyl, butyl, n-butyl, i-butyl, tert.-butyl, pentyl or hexyl.

38. A process according to claim 33, wherein R⁴ is methyl, ethyl, or benzyl unsubstituted or monosubstituted with OCH₃, CH₃, OH, SH, CF₃, F, Cl, Br or I.

39. A process according to claim 8, wherein the thiolate is used stoichiometrically, and an adduct of the thiolate to the compound of Formula 30 is formed, and wherein chlorotrimethylsilane (TMSCl) is used to scavenge the adduct, forming an enol ether.

40. A process according to claim 39, wherein the enol ether is further protonated by a chiral proton donor R*-H.

41. A process according to claim 1, wherein the compound of formula 30 is modified before reaction I with a sterically demanding group.

42. A process according to claim 41, wherein the sterically demanding group is t-Butyldimethylsiloxy (TBDMS).

43. A process according to claim 1, wherein the compound of formula 31 is 3-ethylsulfanyl-2-formylamino-3-methyloctanoic acid ethyl ester or 3-benzylsulfanyl-2-formylamino-3-methyloctanoic acid ethyl ester, the compound of formula 30 is 2-formylamino-3-methyl-2-octenoic acid ethyl ester, and R₄SH is ethyl mercaptan or benzyl mercaptan.

44. A compound of formula 31 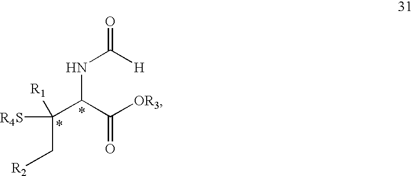
in which
R¹, R² and R³ are independently of one another a C₁₋₁₀ alkyl, saturated or unsaturated, branched or unbranched, mono- or polysubstituted or unsubstituted;
* indicates a stereoselective center,
R⁴ is C₁₋₁₀ alkyl, saturated or unsaturated, branched or unbranched, mono- or polysubstituted or unsubstituted; C₃₋₈ cycloalkyl, saturated or unsaturated, unsubstituted or mono- or polysubstituted; aryl or heteroaryl, in each case unsubstituted or mono- or polysubstituted; or aryl, C₃₋₈ cycloalkyl or heteroaryl, in each case unsubstituted or mono- or polysubstituted, attached via saturated or unsaturated C₁₋₃ alkyl;
in the form of a racemate, an enantiomer, or diastereomer thereof; or a mixture of the enantiomers or diastereomers thereof; or in the form of a physiologically acceptable acidic or basic salt thereof, or in the form of a free acid or base.

45. A compound according to claim 44, in the form of a salt thereof with a cation or a base, or a salt with a anion or an acid.

46. A compound according to claim 44, wherein R¹ is C₁₋₆ alkyl, saturated or unsaturated, branched or unbranched, mono- or polysubstituted or unsubstituted; and R² is C₂₋₉ alkyl, saturated or unsaturated, branched or unbranched, mono- or polysubstituted or unsubstituted.

47. A compound according to claim 46, wherein R¹ is C₁₋₂ alkyl, mono- or polysubstituted or unsubstituted, and R² is C₂₋₉ alkyl, saturated or unsaturated, branched or unbranched, mono- or polysubstituted or unsubstituted.

48. A compound according to claim 46, wherein R¹ is methyl or ethyl.

49. A compound according to claim 46, wherein R² is C₂₋₇ alkyl.

50. A compound according to claim 46, wherein R² is ethyl, propyl, n-propyl, i-propyl, butyl, n-butyl, i-butyl, tert.-butyl, pentyl, hexyl or heptyl.

51. A compound according to claim 44, wherein R¹ is methyl and R² is n-butyl.

52. A compound according to claim 44, wherein R³ is C₁₋₃ alkyl, saturated or unsaturated, branched or unbranched, mono- or polysubstituted or unsubstituted.

53. A compound according to claim 52, wherein R³ is methyl or ethyl.

54. A compound according to claim 44, wherein R⁴ is C₁₋₆ alkyl, saturated or unsaturated, branched or unbranched, mono- or polysubstituted or unsubstituted; phenyl or thiophenyl, unsubstituted or mono substituted; or unsubstituted or mono substituted phenyl attached via CH₃.

55. A compound according to claim 54, wherein R⁴ is phenyl or thiophenyl monosubstituted with OCH₃, CH₃, OH, SH, CF₃, F, Cl, Br or I.

56. A compound according to claim 54, wherein R⁴ is phenyl monosubstituted with OCH₃, CH₃, OH, SH, CF₃, F, Cl, Br or I, attached via CH₃.

57. A compound according to claim 54, wherein R⁴ is saturated, unbranched and unsubstituted, C₁₋₆ alkyl.

58. A compound according to claim 55, wherein R⁴ is methyl, ethyl, propyl, n-propyl, i-propyl, butyl, n-butyl, i-butyl, tert.-butyl, pentyl or hexyl.

59. A compound according to claim 54, wherein R⁴ is methyl, ethyl, or benzyl unsubstituted or monosubstituted with OCH₃, CH₃, OH, SH, CF₃, F, Cl, Br or I.

60. A compound according to claim 1, which is 3-ethylsulfanyl-2-formylamino-3-methyloctanoic acid ethyl ester or 3-benzylsulfanyl-2-formylamino-3-methyloctanoic acid ethyl ester.
